(19) United States
(12) Patent Application Publication
(10) Pub. No.: US 2007/0203098 A1
(43) Pub. Date: Aug. 30, 2007

(54) Pten Inhibitors

(75) Inventors: Joseph R. Garlich, Westfield, IN (US); Donald L. Durden, Decatur, GA (US); Taxiarchis M. Georgiadis, Carmel, IN (US); Jingdong Su, Westfield, IN (US); Xiaodong Peng, Carmel, IN (US); Tim C. Smith, Indianapolis, IN (US)

Correspondence Address: POLSINELLI SHALTON FLANIGAN SUELTHAUS PC, 700 W. 47TH STREET, SUITE 1000, KANSAS CITY, MO, 64112-1802, US

(73) Assignee: Semafore Pharmaceuticals, Inc., Indianapolis, IN (US)

(21) Appl. No.: 10/599,748

(22) PCT Filed: Apr. 6, 2005
(86) PCT No.: PCT/US05/11626

§ 371 Date: Oct. 6, 2006

Related U.S. Application Data
(60) Provisional application No. 60/559,802, filed on Apr. 6, 2004.
(60) Provisional application No. 60/590,043, filed on Jul. 20, 2004.
(60) Provisional application No. 60/625,871, filed on Nov. 8, 2004.

(51) Int. Cl. A61K 31/66; A61K 31/122; A61K 31/13; A61K 31/132; A61K 31/136; A61K 31/40; A61K 31/4192; A61K 31/4245; A61K 31/4375; A61K 31/44

(52) U.S. Cl. 514/75; 514/292; 514/341; 514/359; 514/364; 514/372; 514/418; 514/616; 514/622; 514/659; 514/682

(57) ABSTRACT

The therapeutic use of inhibitors of PTEN activity in the treatment of PTEN-mediated diseases, conditions, and injuries is disclosed.

### CROSS REFERENCE TO RELATED APPLICATIONS

This application claims the benefit of U.S. Provisional Application No. 60/559,802, filed Apr. 6, 2004, U.S. Provisional Application No. 60/590,043, filed Jul. 20, 2004, and U.S. Provisional Application No. 60/625,871, filed Nov. 8, 2004.

### FIELD OF THE INVENTION

The present invention relates to the inhibition of PTEN and therapeutic used thereof.

### BACKGROUND OF THE INVENTION

Cellular processes are to some extent controlled by cycles of phosphorylation and dephosphorylation involving lipids and proteins. PTEN (phosphatase located on chromosome 10) is a dual specificity phosphatase that dephosphorylates phosphatidylinositol 3,4,5 phosphate [Ptlins(3,4,5)P3], which is an important lipid secondary messenger. PTEN is a pivotal signaling molecule modulating a wide variety of cellular processes, including angiogenesis and apoptotic cell death. PTEN coordinates the balance between cell proliferation, angiogenesis and cell death (apoptosis).

PTEN activity leads to activation of the nuclear tumor suppressor protein p53, which leads to apoptosis under conditions of stress. Inhibition of PTEN, thereby increasing levels of PIP3, prevents apoptosis and promotes cell survival. Inhibitors of PTEN could therefore be used to protect important cell populations under conditions of genotoxic or environmental stress.

A number of classes of compound have been identified as PTEN inhibitors, such as bisperoxovanadium compounds [Schmid, A. C. et al., FEBS Lett 2004, 566, (1-3), 35-8] and antisense oligonucleotides [Butler, M et al. Diabetes 2002, 51, (4), 1028-34] but these suffer from properties that make them unlikely to be clinically useful. In addition there have been a number of small molecules claimed to inhibit PTEN including thioredoxin-1 [Meuillet, E. J. et al, Arch. Biochem and Biophys 2004, 429, (2), 123-33], indolecarboxylic acid salts [Fujii, N. et al. J Am Chem Soc 2003, 125, (40), 12074-5], and nonenal [Salsman, S. J. H., et al. 2003; Abstract #3470, American Association for Cancer Research 2003], but none of these directly inhibit PTEN's dephosphorylation ability with a reversible antagonist mode of action and thus are unlikely to be developed into useful small molecule drugs. Other phosphatase inhibitors are known, such as the bisphosphonate alendronate, but they have not been demonstrated to have PTEN activity. Considering the importance of the role of PTEN in apoptosis and angiogenesis, there remains a need in the art for PTEN inhibitors.

### SUMMARY OF THE INVENTION

The present invention is related to a method of protecting a patient from one or more treatments that trigger apoptosis. The patient may be administered a composition comprising a pharmaceutically acceptable amount of a PTEN inhibitor selected from compounds I-XIV. The treatment may be a cancer treatment. The PTEN inhibitor may be administered prior to, together with, or after a treatment for the cancer. The treatment may be chemotherapy or radiation therapy.

The present invention is also related to a method of treating a patient suffering from damage to normal tissue attributable to stress. The patient may be administered a composition comprising a pharmaceutically acceptable amount of a PTEN inhibitor selected from compounds I-XIV. The agent may be administered prior to, together with, or after a treatment for a disease suffered by the patient.

The present invention is also related to a method of sensitizing cancer cells to an inhibitor of the PI3 kinase pathway. The patient may be administered a composition comprising a pharmaceutically acceptable amount of a PTEN inhibitor selected from compounds I-XIV.

The present invention is also related to a method of treating apoptosis associated with a medical procedure. The patient may be administered a composition comprising a pharmaceutically acceptable amount of a PTEN inhibitor selected from compounds I-XIV. The present invention is also related to a method of sensitizing cancer stem cells to conventional treatment. The patient may be administered a composition comprising a pharmaceutically acceptable amount of a PTEN inhibitor selected from compounds I-XIV. The present invention is also related to a method of inducing or stimulating angiogenesis in a patient in need thereof. The patient may be administered a composition comprising a pharmaceutically acceptable amount of a PTEN inhibitor selected from compounds I-XIV.

### DETAILED DESCRIPTION

Before the present compounds, products and compositions and methods are disclosed and described in detail, it is to be understood that the terminology used herein is for the purpose of describing particular embodiments only and is not intended to be limiting. It must be noted that, as used in the specification and the appended claims, the singular forms “a,” “an” and “the” include plural referents unless the context clearly dictates otherwise.

1. Definitions

The term “branched” as used herein refers to a group containing from 1 to 24 backbone atoms wherein the backbone chain of the group contains one or more subordinate branches from the main chain. Preferred branched groups herein contain from 1 to 12 backbone atoms. Examples of branched groups include, but are not limited to, isobutyl, t-butyl, isopropyl, —CH₂CH₂CH(CH3)CH₂CH₃, —CH₂CH(CH₂CH₃)CH₂CH₃, —CH₂CH₂C(CH₃₎₂CH₃, —CH₂CH₂C(CH₃)₃ and the like.

The term “unbranched” as used herein refers to a group containing from 1 to 24 backbone atoms wherein the backbone chain of the group extends in a direct line. Preferred unbranched groups herein contain from 1 to 12 backbone atoms.

The term “cyclic” or “cyclo” as used herein alone or in combination refers to a group having one or more closed rings, whether unsaturated or saturated, possessing rings of from 3 to 12 backbone atoms, preferably 3 to 7 backbone atoms.

The term “lower” as used herein refers to a group with 1 to 6 backbone atoms.

The term “saturated” as used herein refers to a group where all available valence bonds of the backbone atoms are attached to other atoms. Representative examples of saturated groups include, but are not limited to, butyl, cyclohexyl, piperidine and the like.

The term “unsaturated” as used herein refers to a group where at least one available valence bond of two adjacent backbone atoms is not attached to other atoms. Representative examples of unsaturated groups include, but are not limited to, —CH₂CH₂CH═CH₂, phenyl, pyrrole and the like.

The term “aliphatic” as used herein refers to an unbranched, branched or cyclic hydrocarbon group, which may be substituted or unsubstituted, and which may be saturated or unsaturated, but which is not aromatic. The term aliphatic further includes aliphatic groups, which comprise oxygen, nitrogen, sulfur or phosphorous atoms replacing one or more carbons of the hydrocarbon backbone.

The term “aromatic” as used herein refers to an unsaturated cyclic hydrocarbon group having 4n+2 delocalized π(pi) electrons, which may be substituted or unsubstituted. The term aromatic further includes aromatic groups, which comprise a nitrogen, oxygen, or sulfur atom replacing one or more carbons of the hydrocarbon backbone. Examples of aromatic groups include, but are not limited to, phenyl, naphthyl, thienyl, furanyl, pyridinyl, (is)oxazoyl and the like.

The term “substituted” as used herein refers to a group having one or more hydrogens or other atoms removed from a carbon or suitable heteroatom and replaced with a further group. Preferred substituted groups herein are substituted with one to five, most preferably one to three substituents. An atom with two substituents is denoted with “di,” whereas an atom with more than two substituents is denoted by “poly.” Representative examples of such substituents include, but are not limited to aliphatic groups, aromatic groups, alkyl, alkenyl, alkynyl, aryl, alkoxy, halo, aryloxy, carbonyl, acryl, cyano, amino, nitro, phosphate-containing groups, sulfur-containing groups, hydroxyl, alkylcarbonyloxy, arylcarbonyloxy, alkoxycarbonyloxy, aryloxycarbonyloxy, alkylcarbonyl, arylcarbonyl, alkoxycarbonyl, aminocarbonyl, alkylaminocarbonyl, dialkylaminocarbonyl, alkylthiocarbonyl, acylamino, amidino, imino, alkylthio, arylthio, thiocarboxylate, alkylsulfinyl, trifluoromethyl, azido, heterocyclyl, alkylaryl, heteroaryl, semicarbazido, thiosemicarbazido, maleimido, oximino, imidate, cycloalkyl, cycloalkylcarbonyl, dialkylamino, arylcycloalkyl, arylcarbonyl, arylalkylcarbonyl, arylcycloalkylcarbonyl, arylphosphinyl, arylalkylphosphinyl, arylcycloalkylphosphinyl, arylphosphonyl, arylalkylphosphonyl, arylcycloalkylphosphonyl, arylsulfonyl, arylalkylsulfonyl, arylcycloalkylsulfonyl, CF₃, combinations thereof, and substitutions thereto.

The term “unsubstituted” as used herein refers to a group that does not have any further groups attached thereto or substituted therefor.

The term “alkyl” as used herein alone or in combination refers to a branched or unbranched, saturated aliphatic group. Representative examples of alkyl groups include, but are not limited to, methyl, ethyl, n-propyl, isopropyl, n-butyl, isobutyl, sec-butyl, tert-butyl, octyl, decyl, tetradecyl, hexadecyl, eicosyl, tetracosyl and the like.

The term “alkenyl” as used herein alone or in combination refers to a branched or unbranched, unsaturated aliphatic group containing at least one carbon-carbon double bond which may occur at any stable point along the chain. Representative examples of alkenyl groups include, but are not limited to, ethenyl, E- and Z-pentenyl, decenyl and the like.

The term “alkynyl” as used herein alone or in combination refers to a branched or unbranched, unsaturated aliphatic group containing at least one carbon-carbon triple bond which may occur at any stable point along the chain. Representative examples of alkynyl groups include, but are not limited to, ethynyl, propynyl, propargyl, butynyl, hexynyl, decynyl and the like.

The term “aryl” as used herein alone or in combination refers to a substituted or unsubstituted aromatic group, which may be optionally fused to other aromatic or non-aromatic cyclic groups. Representative examples of aryl groups include, but are not limited to, phenyl, pyridyl-, furazan, benzyl, naphthyl, benzylidine, xylyl, styrene, styryl, phenethyl, phenylene, benzenetriyl and the like.

The term “alkoxy” as used herein alone or in combination refers to an alkyl, alkenyl or alkynyl group bound through a single terminal ether linkage. Examples of alkoxy groups include, but are not limited to, methoxy, ethoxy, n-propoxy, iso-propoxy, n-butoxy, 2-butoxy, tert-butoxy, n-pentoxy, 2-pentoxy, 3-pentoxy, isopentoxy, neopentoxy, n-hexoxy, 2-hexoxy, 3-hexoxy, 3-methylpentoxy, fluoromethoxy, difluoromethoxy, trifluoromethoxy, chloromethoxy, dichloromethoxy, and trichloromethoxy.

The term “aryloxy” as used herein alone or in combination refers to an aryl group bound through a single terminal ether linkage.

The term “halogen,” “halide” or “halo” as used herein alone or in combination refers to fluorine “F”, chlorine “Cl”, bromine “Br”, iodine “I”, and astatine “At”. Representative examples of halo groups include, but are not limited to, chloroacetamido, bromoacetamido, idoacetamido and the like.

The term “hetero” as used herein combination refers to a group that includes one or more atoms of any element other than carbon or hydrogen. Representative examples of hetero groups include, but are not limited to, those groups that contain heteroatoms including, but not limited to, nitrogen, oxygen, sulfur and phosphorus.

The term “heterocycle” as used herein refers to a cyclic group containing a heteroatom. Representative examples of heterocycles include, but are not limited to, pyridine, piperadine, pyrimidine, pyridazine, piperazine, pyrrole, pyrrolidinone, pyrrolidine, morpholine, thiomorpholine, indole, furazan, isoindole, imidazole, triazole, tetrazole, furan, benzofuran, dibenzofuran, thiophene, thiazole, benzothiazole, benzoxazole, benzothiophene, quinoline, isoquinoline, azapine, naphthopyran, furanobenzopyranone and the like.

The term “carbonyl” or “carboxy” as used herein alone or in combination refers to a group that contains a carbon-oxygen double bond. Representative examples of groups which contain a carbonyl include, but are not limited to, aldehydes (i.e., formyls), ketones (i.e., acyls), carboxylic acids (i.e., carboxyls), amides (i.e., amidos), imides (i.e., imidos), esters, anhydrides and the like.

The term “acryl” as used herein alone or in combination refers to a group represented by CH₂═C(Q)C(O)O— where Q is an aliphatic or aromatic group.

The term “cyano,” “cyanate,” or “cyanide” as used herein alone or in combination refers to a carbon-nitrogen double bond or carbon-nitrogen triple bond. Representative examples of cyano groups include, but are not limited to, isocyanate, isothiocyanate and the like.

The term “amino” as used herein alone or in combination refers to a group containing a backbone nitrogen atom. Representative examples of amino groups include, but are not limited to, alkylamino, dialkylamino, arylamino, diarylamino, alkylarylamino, alkylcarbonylamino, arylcarbonylamino, carbamoyl, ureido and the like.

The term “phosphate-containing group” as used herein refers to a group containing at least one phosphorous atom in an oxidized state. Representative examples include, but are not limited to, phosphonic acids, phosphinic acids, phosphate esters, phosphinidenes, phosphinos, phosphinyls, phosphinylidenes, phosphos, phosphonos, phosphoranyls, phosphoranylidenes, phosphorosos and the like.

The term “sulfur-containing group” as used herein refers to a group containing a sulfur atom. Representative examples include, but are not limited to, sulfhydryls, sulfenos, sulfinos, sulfinyls, sulfos, sulfonyls, thios, thioxos and the like.

The term “optional” or “optionally” as used herein means that the subsequently described event or circumstance may or may not occur, and that the description includes instances where said event or circumstance occurs and instances where it does not. For example, the phrase “optionally substituted alkyl” means that the alkyl group may or may not be substituted and that the description includes both unsubstituted alkyl and alkyl where there is a substitution.

The term “effective amount,” when used in reference to a compound, product, or composition as provided herein, means a sufficient amount of the compound, product or composition to provide the desired result. The exact amount required will vary depending on the particular compound, product or composition used, its mode of administration and the like. Thus, it is not always possible to specify an exact “effective amount.” However, an appropriate effective amount may be determined by one of ordinary skill in the art informed by the instant disclosure using only routine experimentation.

The term “suitable” as used herein refers to a group that is compatible with the compounds, products, or compositions as provided herein for the stated purpose. Suitability for the stated purpose may be determined by one of ordinary skill in the art using only routine experimentation.

2. Compounds

The present invention is related to compounds that are inhibitors of the dephosphorylation abilities of the enzyme PTEN (“PTEN Inhibitors”). The compounds may be used to inhibit PTEN in a patient, which may have any of a number of beneficial purposes as described herein.

a. Ascorbic Acid-Based PTEN Inhibitors

The compound of the present invention may be an ascorbic acid derivative or dehydroascorbic acid derivative selected from the following: 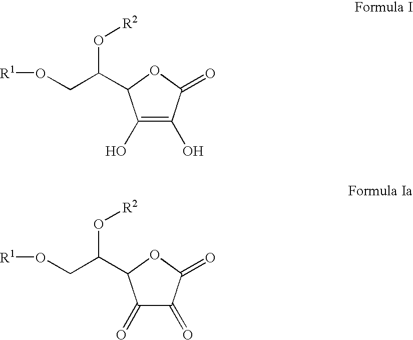
wherein,
R¹ represents H, C¹-C³ alkyl, aryl, alkylaryl, (CH₂)ₙCOXR³, (CH₂)ₙXCOR³, (CH₂)ₙCOR³, (CH₂)ₙSO₂R³, (CH₂)ₙXR³, (CH₂)ₙSO₂X—R³, (CH₂)ₙXSO₂R³, (CH₂)ₙNR³R⁴, or (CH₂)ₙCO(CH₂)ₘXR³;
R² represents H, C¹-C³ alkyl, aryl, alkylaryl, (CH₂)ₙCOX—R³, (CH₂)ₙXCOR³, (CH₂)ₙCOR³, (CH₂)ₙSO₂R³, (CH₂)ₙXR³, (CH₂)ₙSO₂X—R³, (CH₂)ₙXSO₂R³, (CH₂)ₙNR³R⁴, or (CH₂)ₙCO(CH₂)ₘXR³;
R³, R⁵ and R⁶independently are H, C¹-C⁴ alkyl, aryl or alkylaryl;
R⁴ represents H, C¹-C⁴ alkyl, aryl, alkylaryl, NHSO₂R⁵, NHCO₂R⁵, or NR⁵R⁶;
m=0 to 3;
n=0 to 3; and
X represents O or NR⁴.

Compounds of Formula I and Ia may have ester linkages at either R¹ or R².

(1) General Synthesis

Both ascorbic acid and dehydroascorbic acid are commercially available and can be easily acylated on the primary and secondary alcohol groups using reactive acid chlorides as is well known in the art for preparing esters from alcohols. The primary alcohol is primarily acylated first and by adjusting the stoichiometry close to one-to-one, preferential acylation may occur to give compounds of Formula I and Formula Ia wherein the R² group is a hydrogen. These types of monoacylated compounds may then be further acylated to provide R2 other than hydrogen by the same chemistry (e.g. acid chlorides or activated esters). Additionally, the chemistry to make derivatives of ascorbic acid and dehydroascorbic acid are well described in the literature (Manfredini et al., J. Med. Chem. 2002, vol. 45, pps. 559-562, Hak Hee Kang, et al., and Bull. Korean Chem. Soc. 2003, vol. 24, No. 8, 1169-1171 and references therein).

b. Triazoles

The compound of the present invention may also be a 1,2,3-triazole, which is described in Olesen et al, WO 02/32896, the contents of which are incorporated herein by reference. The compound may be of the formula: 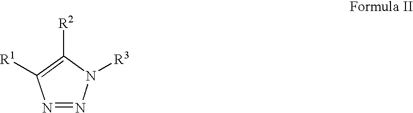
wherein,
R¹ represents H, C¹-C⁴ alkyl, aryl, alkylaryl, COXR², COR², SO₂XR², SO₂R²;
R² represents H, C¹-C⁴ alkyl, aryl, alkylaryl, (CH₂)ₙCOXR⁴, (CH₂)ₙXCOR⁴, (CH₂)ₙX R⁴, (CH₂)ₙSO₂XR⁴, (CH₂)ₙXSO₂R⁴, NHSO₂R⁴, NHCOR⁴, NHCO₂R⁴, NHCOCO₂R⁴, or NR⁴R⁵;
R³ represents H, C¹-C⁴ alkyl, aryl, alkylaryl, (CH₂)ₙCOXR⁴, (CH₂)ₙXCOR⁴, (CH₂)ₙX R⁴, (CH₂)ₙSO₂XR⁴, (CH₂)ₙXSO₂R⁴, NHSO₂R⁴, NHCOR⁴, NHCO₂R⁴, NHCOCO₂R⁴, or NR⁴R⁵;
R⁴ represents H, C¹-C⁴ alkyl, aryl, or alkylaryl;
R⁵ represents H, C¹-C⁴ alkyl, aryl, alkylaryl, NHSO₂R⁶, NHCOR⁶, NHCO₂R⁶, NR⁶R⁷, or N═C(R⁶R⁷);
R⁶ represents H, C¹-C⁴ alkyl, aryl, or alkylaryl;
R⁷ represents H, C¹-C⁴ alkyl, aryl, or alkylaryl;
n=0-3; and
X represents O or NR⁵.

The Compound of Formula II may be the following: 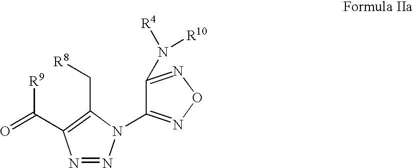
wherein,
R⁸ represents (CH₂)ₙXR⁴, or (CH₂)ₙSR⁴;
R⁹ represents NHNHSO₂aryl, NHNHCOaryl, or NHN═C(R⁶R⁷); and
R¹⁰ represents H, C¹-C⁴ alkyl, aryl, alkylaryl, SO₂R⁶, COR⁶, or CO₂R⁶.

(1) General Synthesis

Compounds of Formula IIA, may be synthesized from the key intermediate 2-1 [Olesen et al., (2002), WO200232896-A, 71pp; Olesen et al., (2003), J Med Chem, 46, 15, 3333-41].

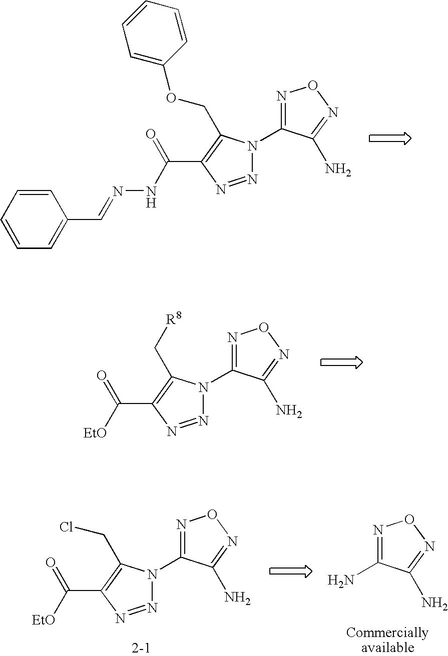

This intermediate ester 2-1 may be synthesized in one step from readily available furazan [Fernandez et al., (2002), Tetrahed. Let., 43, 4741-4745]. The aliphatic chloride may be displaced by a variety of nucleophiles (R⁸). The ester group may be converted to an activated ester and reacted with the aforementioned nucleophiles (R⁹) or the ester group can be reacted directly with the nucleophiles that make up the R⁹ substituent.

c. Diamides

The compound may also be a diamide of the formula: 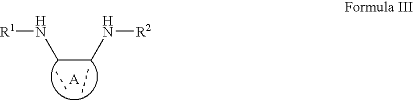
wherein,
A is a five or six member ring;
R¹ represents H, C¹-C³ alkyl, aryl, alkylaryl, (CH₂)ₙCOXR³, (CH₂)ₙXCOR³, (CH₂)ₙCOR³, (CH₂)ₙSO₂R³, (CH₂)ₙXR³, (CH₂)ₙSO₂XR³, (CH₂)ₙXSO₂R³, NHSO₂R³, NHCO₂R³, NHCOR³, NHCO₂R³, NHCOCO₂R³, NR³R⁴, or (CH₂)ₙCO(CH₂)ₘXR³;
R² represents H, C¹-C³ alkyl, aryl, alkylaryl, (CH₂)ₙCOXR³, (CH₂)ₙXCOR³, (CH₂)ₙCOR³, (CH₂)ₙSO₂R³, (CH₂)ₙXR³, (CH₂)ₙSO₂XR³, (CH₂)ₙXSO₂R³; NHSO₂R³, NHCO₂R³, NHCOR³, NHCO₂R³, NHCOCO₂R³, NR³R⁴, or (CH₂)ₙCO(CH₂)ₘXR³;
R³ represents H, C¹-C⁴ alkyl, aryl, or alkylaryl;
R⁴ represents H, C¹-C⁴ alkyl, aryl, alkylaryl, NHSO₂R⁵, NHCO₂R⁵, or NR⁵R⁶;
R⁵ represents H, C¹-C⁴ alkyl, aryl, or alkylaryl;
R⁶ represents H, C¹-C⁴ alkyl, aryl, or alkylaryl;
n=0-3;
m=0-3; and
X represents O, or NR⁴.

Ring A may be saturated, unsaturated, or aromatic, and may optionally comprise N and O. Preferred compounds of formula III are those wherein ring A is selected from heterocyclic ring systems, especially vicinally substituted pyridines, pyrimidines, furazans, imidazoles, pyrrazoles, furans, thiazoles, and oxazoles, as well as their saturated analogs; other preferred compounds of formula III are those wherein ring A comprises an all carbon aromatic rings, such as substituted and unsubstituted phenyl, and their saturated analogs.

The Compound of Formula III may comprise a ring A selected from the following:

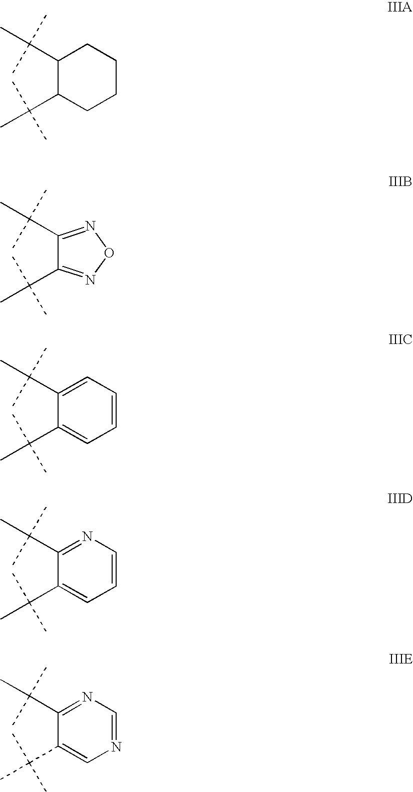

The Compound of Formula III comprising a ring A selected from IIIA, IIIB, IIIC, IIID, IIIE may further comprise the following:
R¹ represents H, C¹-C³ alkyl, aryl, alkylaryl, (CH₂)ₙCOR³, or (CH₂)ₙSO₂R³;
R³ represents H, C¹-C⁴ alkyl, aryl, or alkylaryl;
R⁷ represents H, C¹-C⁴ alkyl, halogens, NO₂, CF₃, aryl, carboxylate, aryloxy, amino, alkylamino, cyano, isocyanate, alkoxycarbonyl, or haloalkyl;
R⁸ represents H, C¹-C⁴ alkyl, halogens, NO₂, CF₃, aryl, carboxylate, aryloxy, amino, alkylamino, cyano, isocyanate, alkoxycarbonyl, or haloalkyl; and
m=1,2,3.
alkylaryl is selected from Formula IIIF or IIIG:

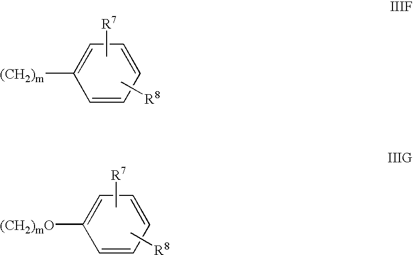

The Compound of Formula III may also be of the formula: 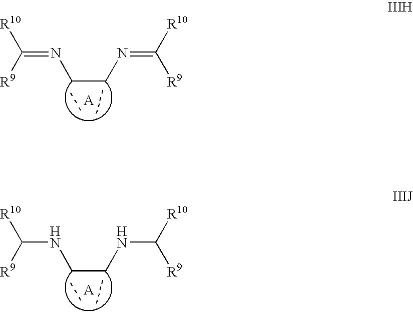
wherein,
A is a five or six member ring;
R⁹ represents H, C¹-C³ alkyl, aryl, alkylaryl, (CH₂)ₙCOXR³, (CH₂)ₙXCOR³, (CH₂)ₙCOR³, CH₂(CH₂)ₙSO₂R³, CH₂(CH₂)ₙXR³, CH₂(CH₂)ₙSO₂XR³, or CH—₂(CH₂)ₙXSO₂R³; and
R¹⁰ represents H, C¹-C³ alkyl, aryl, alkylaryl, (CH₂)ₙCOXR³, (CH₂)ₙXCOR³, (CH₂)ₙCOR³, CH₂(CH₂)ₙSO₂R³, CH₂(CH₂)ₙXR³, CH₂(CH₂)ₙSO₂XR³, or CH—₂(CH₂)ₙXSO₂R³.

And where R³, X, and n are as described for Formula III.

Ring A of Compounds IIIH and IIIJ may be saturated, unsaturated or aromatic, and may optional be substituted with C and N.

(1) General Synthesis

(a) Amide Linkers

Compound III may be synthesized as a substituted 1,2 diamino aryl ring or 1,2 disubstituted aliphatic ring. The core aromatic ring may be any 5 and 6 member aromatic or hetero aromatic ring. Specific cores may be derived from substituted and unsubstituted diamino benzenes, benzenes, pyridines pyrimidines, furazans, and other aromatic and heteroaromatic rings. The furazan core diamide series may be synthesized from commercially available (Acros Organics) 3,4-diamino furazan [Fernandez et al. (2002), Tetrahed. Let., 43, 4741-4745] coupled to a suitable activated ester. The aliphatic rings may be 1,2 diamino cyclopentane or 1,2 diamino cyclohexane. As shown below, phenoxy acetyl chlorides and other acid chlorides may react with the aryl amine core to yield the desired products [Sorba et al., Archiv der Pharmazie (1989), 322(9), 579-80]. Altering the stoichiometry of this reaction may yield the mono substituted core ring (3-1) or the disubstituted core ring (3-2).

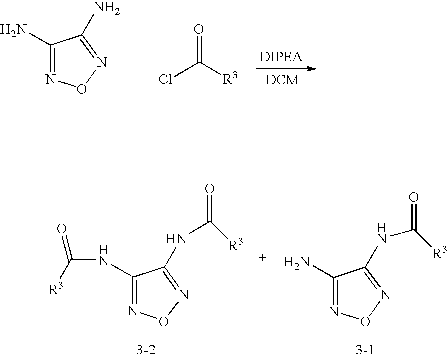

Based on the biological activity revealed herein, the activated esters or acid chlorides may contain an aryl ring attached via a suitably length tether. The acid chloride (or activated ester) may be derived from commercially available benzoic acids, cinnamic acids, hydrocinnamic acids, phenoxy acetic acids, phenylpropionic acid, phenyl isocyanates, benzyloxyacetic acids. The aromatic rings in the tether may consist of thiophenes, pyridines, pyrimidines, phenyl, and furans. The tether linking the aromatic moiety to the core furazan may be freely rotating (e.g., aliphatic) or constrained with a double or triple bond. In addition, the corresponding sulfamide compounds may be synthesized.

(b) Aliphatic linkers

The linker between the furazan core and the aromatic ring containing groups may be an aliphatic amine [Zelenin and Trudell (1997), J. Heterocycl. Chem., 34, 3 1057-1060] via a two step reductive alkylation from the corresponding aldehyde or ketone group. Altering the stoichiometry of this reaction will yield the mono substituted core ring or the disubstituted core ring.

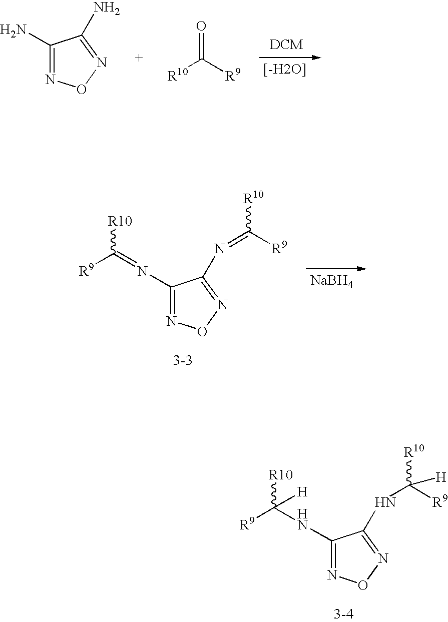

d. Aryl Imidazole Carbonyl Derivatives

The compound of the present invention may also be of the formula: 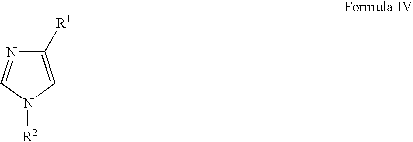
wherein,
R¹ represents H, C¹-C⁴ alkyl, aryl, alkylaryl, (CH₂)ₙCOXR³, (CH₂)ₘXCOR³, (CH₂)ₘXR³, (CH₂)ₙCOR³, (CH₂)ₙSO₂XR³, or (CH₂)ₘXSO₂R³;
R² represents H, C¹-C⁴ alkyl, aryl, or alkylaryl;
R³ represents H, C¹-C³ alkyl, aryl, or alkylaryl;
R⁴ represents H, C¹-C⁴ alkyl, aryl, alkylaryl, NHSO₂R⁵, NHCO₂R⁵, N═C(R⁵R⁶), or NR⁵R⁶;
R⁵ represents H, C¹-C⁴ alkyl, aryl, or alkylaryl;
R⁶ represents H, C¹-C⁴ alkyl, aryl, or alkylaryl;
m=1-3;
n=0-3; and
X represents O, NR⁴.

Compounds of formula IV may be of the formula: 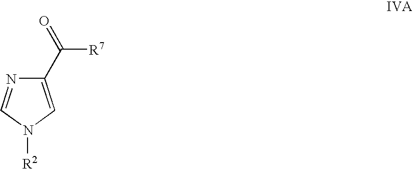
wherein, R⁷ represents XR⁴.

Compounds of formula IV may also be of the formula: 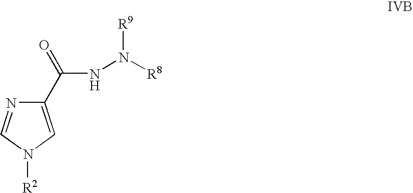
wherein
R⁸ represents H, C¹-C⁴ alkyl, aryl, alkylaryl, (CH₂)ₙCOX—R³, (CH₂)ₙXCOR³, (CH₂)ₙX—R³, (CH₂)ₙCOR³, (CH₂)ₙSO₂XR³, or (CH₂)ₙXSO₂R³; and
R⁹ represents H, C¹-C⁴ alkyl, aryl, alkylaryl.

Compounds of Formula IVB may also be selected from the following:

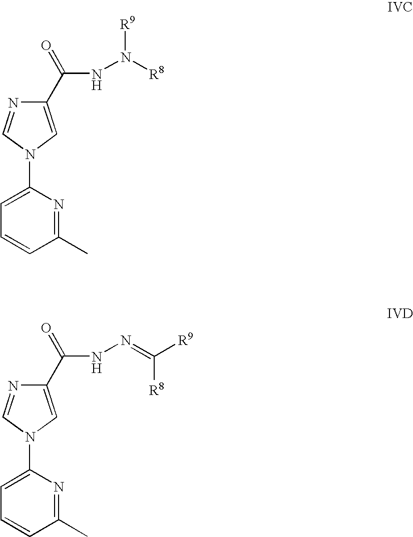

(1) General Synthesis of Aryl Imidazole Carbonyl Derivatives

The Compounds of Formula IV may be synthesized using an imidazole carbonyl core. Methyl 4-imidazole carboxylate may be coupled with aryl halides via copper-catalyzed N-arylation [Kiyomori et al. (1999), Tetrahed. Lett., 40, 14 2657-2660]. Alternatively, the use of aryl boronic acid may be utilized for the coupling reaction [Collman and Zhong (2000) Org. Lett. 2000 2(9), 1233-1236 and Combs et al. (1999), Tetrahed. Lett. 40(-9] 1623-1626]. Using standard acylation/alkylation chemistry, hydrazine may be coupled to the imidazole moiety on one end, and with various acyl/alkyl groups on the other, allowing for fine tuning electronic properties, bulk, and overall length of the inhibitor.

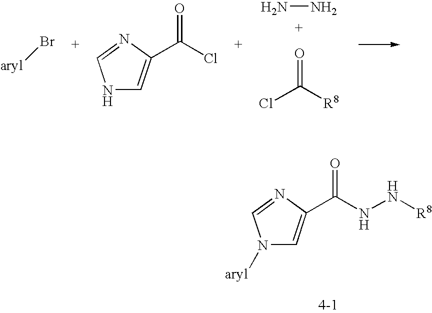

Furthermore, treatment of the hydrazide intermediate, 4-2, with aldehydes or ketones may produce the corresponding imine 4-3.

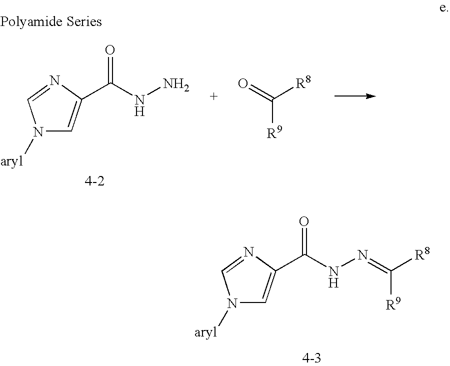

The compound of the present invention may also be a polyamide selected from the following:

| Structure | SF Number |
|---|---|
| 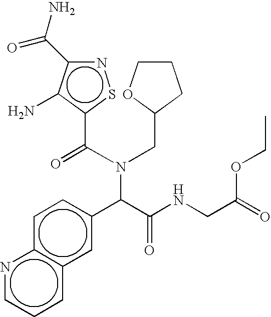 | SF1566-000 |
| 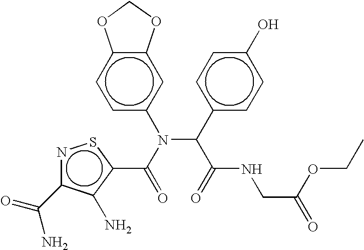 | SF1552-000 |
| 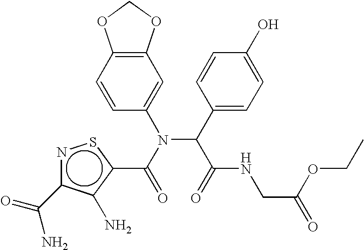 | SF1560-000 |
| 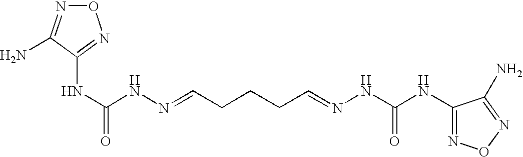 | SF1567-000 |
| 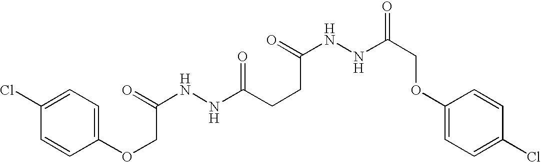 | SF1516-000 |

f. Commercially Available Known PTP Inhibitors for PTEN Inhibition

The compound of the present invention may also be selected from the following:

| | Compound | |
|---|---|---|
| STRUCTURE | Name | SF Number |
| 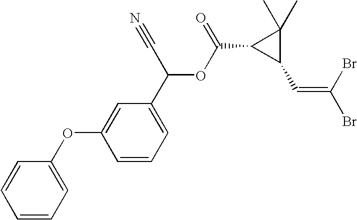 | Deltamethrin; (S)-a-Cyano-3- phenoxybenzyl(1R)- cis-3-(2,2- dibromovinyl)-2,2- dimethylcyclopropane- carboxylate | SF1667-000 |
| 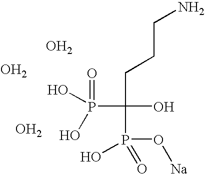 | Alendronate, Sodium, Trihydrate | SF1669-100 |
| 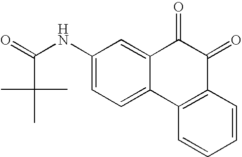 | N-(9,10-Dioxo- 9,10-dihydro- phenanthren-2-yl)- 2,2-dimethyl- propionamide | SF1670-000 |
| 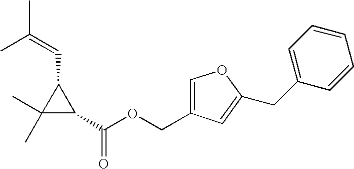 | 5-Benzyl-3- furylmethyl (1R,S)-cis,trans- chrysanthemate | SF1671-000 |
| 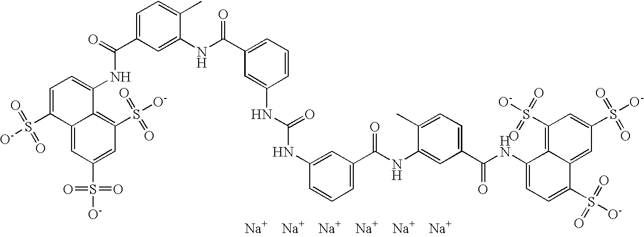 | Suramin, Sodium Salt; 8,8′- [carbonylbis[imino-3,1- phenylenecarbonylimino (4-methyl-3,1- phenylene)carbonyl- imino]]bis-, hexasodium salt | SF1672-100 |
| 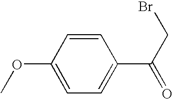 | 4-Methoxyphenacyl Bromide | SF1673-000 |
| 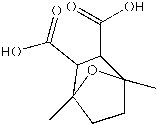 | 1,4-Dimethylendothall; 1,4-Dimethyl-7- oxabicyclo[2.2.1]heptane-2,3- dicarboxylic Acid | SF1676-000 |
| 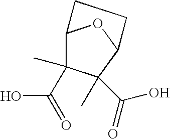 | Cantharidic Acid; 2,3-dimethyl- 7-oxa- bicyclo[2.2.1]heptane-2,3- dicarboxylic acid | SF1679-000 |
| 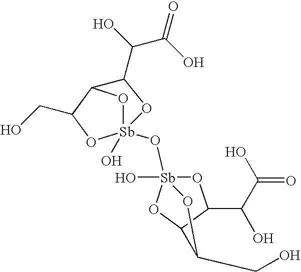 | Sodium Stibogluconate; Antimony Sodium Gluconate | SF1680-000 |
| 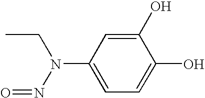 | 3,4-Dephostatin, Ethyl- | SF1681-000 |
| 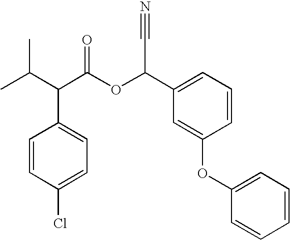 | Fenvalerate; a-Cyano-3- phenoxybenzyl-a- (4-chlorophenyl)isovalerate | SF1683-000 |
| 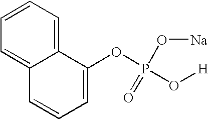 | α-Naphthyl Acid Phosphate, Monosodium Salt | SF1684-100 |
| 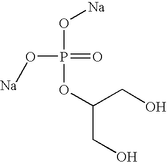 | β-Glycerophosphate, Disodium Salt, Pentahydrate | SF1685-100 |
| 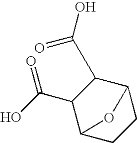 | Endothall; 7-Oxabicyclo[2.2.1]heptane-2,3- dicarboxylic Acid | SF1686-000 |
| 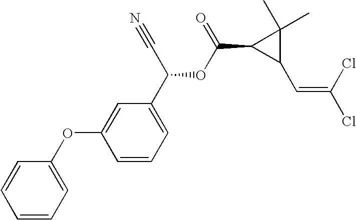 | Cypermethrin; (R,S)-α- Cyano-3-phenoxybenzyl-3- (2,2-dichlorovinyl)-2,2- dimethylcyclopropane- carboxylate; (1R)-(R)- cyano(3-phenoxyphenyl) methyl 3-(2,2- dichlorovinyl)-2,2- dimethylcyclopropane- carboxylate | SF1687-000 |

g. Phenanthrolines

The compound of the present invention may also be a substituted 1,10-phenanthroline-5,6-dione of the formula: 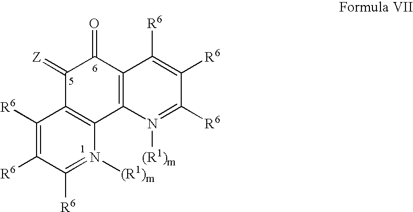
wherein,
R¹ represents O, C¹-C⁴ alkyl, (CH₂)ₙCOXR², (CH₂)ₙXCOR², (CH₂)ₙXR², (CH₂)ₙCOR², (CH₂)ₙSO₂XR², (CH₂)ₙXSO₂R², or (CH₂)ₙSO₂R²;
R² represents H, C¹-C⁴ alkyl, aryl, alkylaryl, NHSO₂R⁴, NHCOR⁴, NHCO₂R⁴, NHCOCO₂R⁴, or NR⁴R⁵;
R³ represents H, C¹-C⁴ alkyl, aryl, alkylaryl, NHSO₂R⁴, NHCOR⁴, NHCO₂R⁴, NHCOCO₂R⁴, or NR⁴R⁵;
R⁴ represents H, C¹-C⁴ alkyl, aryl, or alkylaryl;
R⁵ represents H, C¹-C⁴ alkyl, aryl, or alkylaryl;
R⁶ at each occurrence is independently selected from hydrogen, halogen, NO₂, NR⁴R¹⁰, C¹-C⁴ alkyl, NH(CH₂)ₚCO(CH₂)_{q}XR², (CH₂)ₚCOXR², (CH₂)ₚXCOR², (CH₂)ₚXR², (CH₂)ₚCOR², (CH₂)ₚSO₂XR², or (CH₂)ₚXSO₂R²;
R⁷ represents H, C¹-C⁴ alkyl, aryl, alkylaryl, SO₂R⁴, NHSO₂R⁴, NHCO₂R⁴, or NR⁸R⁹;
R⁸ represents independently H, C¹-C⁴ alkyl, aryl, alkylaryl, (CH₂)ₙCOXR², or (CH₂)ₙXR²;
R⁹ represents independently H, C¹-C⁴ alkyl, aryl, alkylaryl, (CH₂)ₙCOXR², (CH₂)ₙXR², (CH₂)ₚCOXR², (CH₂)ₚXCOR², (CH₂)ₚXR², (CH₂)ₚCOR², (CH₂)ₚSO₂XR², (CH₂)ₚXSO₂R², or (CH₂)ₚSO₂R²;
R10 represents H, C1-C4 alkyl R7=H, C1-C4 alkyl, aryl, alkylaryl, SO2R4, NHSO2R4, NHCO2R4, or NR8R9;
m represents independently 0 or 1;
n=1-5;
p=0-5;
q=0-5;
X represents O or NR³; and
Z=O or NR⁷.

The nitrogen in the ring of compound of Formula VII may be neutral. The nitrogen may also be charged when bound to an R¹ group (quaternary salt) in the case where at least one m=1. The compound of the present invention may also be selected from Formula VIIa, VIIb, and VIIc:

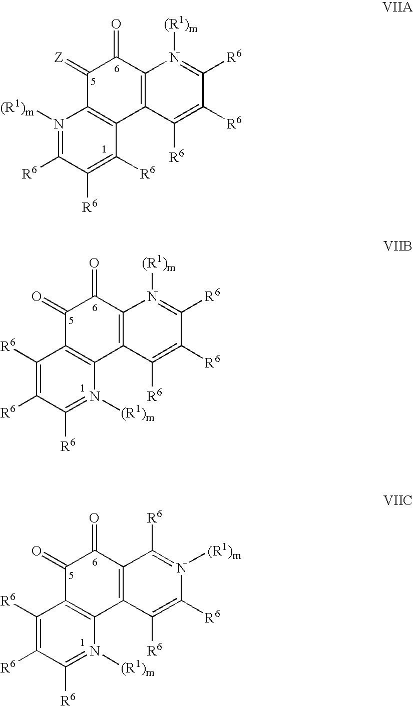

(1) General Synthesis

1,10-Phenanthrolines may be brominated to yield either the mono or dibromo adduct [Boldron et al., (2001), Synlett, 10, 1629-1631]. The bromo arene can be reacted to yield the corresponding substituted aromatic ring system.

1,10-Phenanthroline may be readily oxidized to the corresponding 1,10-phenanthroline-5,6-dione(5-9) using known methodology [Hiort et al., (1993), J. Am. Chem. Soc., 115, 9, 3448-3454; and Lopez et al., (1996), Tetrahed. Lett., 37, 31, 5437-5440].

1,10-phenanthroline (7-1) may be converted to the corresponding phenanthrolinium salt (7-2) using MeI or CF₃SO₃CH₃ in CH₂Cl₂ [Geisler et al., (2003), Synthesis, 8, 1215-1220] followed by oxidation to the corresponding 1,10-Phenanthroline-5,6-dione (7-3).

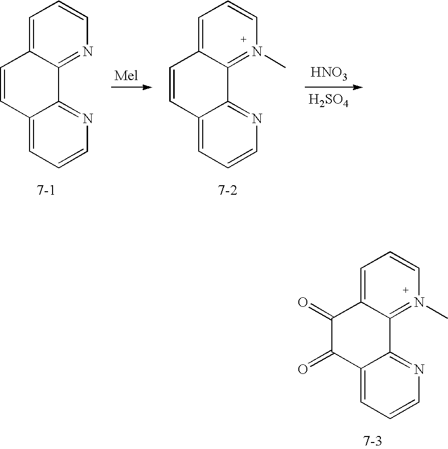

In addition, the nitrogens in the phenanthrolines 7-1, may be oxidixed with H₂O₂ in benzene-AcOH at 80° C. to 7-4, followed by oxidation to 7-5.

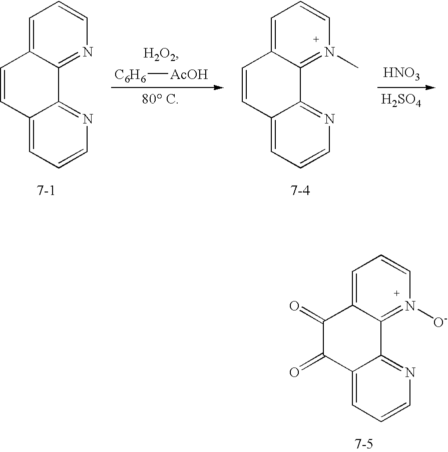

1,8-phenanthroline-5,6-diones of Formula 7-8 may be synthesized from the corresponding 5-aminoisoquinoline, 7-6, [Jastrzebska-Glapa and Mlochowski, (1976), Roczniki Chemii, 50, 5, 987-91; and Markees, (1983), Helvetica Chimica Acta, 66, 2, 620-6] reacted with acrolein to yield the 1,8 phenanthroline 7-7. This may readily be oxidized to the desired compound of Formula 7-8.

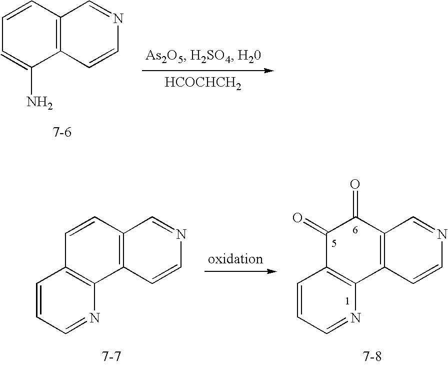

The 1,10-phenanthroline-5,6-diones, 7-9, may be reacted with aromatic or aliphatic amines and upon loss of water yield 7-10. The 1,10-phenanthroline-5,6-diones may also be reacted with reactive halo compounds under forcing conditions to prepare the quaternary salts of Formula VII.

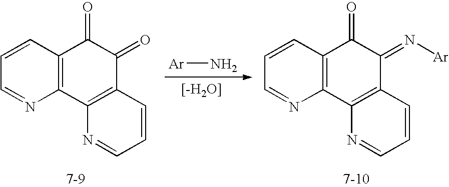

h. Phenanthrene Diones

The compound of the present invention may also be a substituted phenanthrene-9,10-diones of the formula: 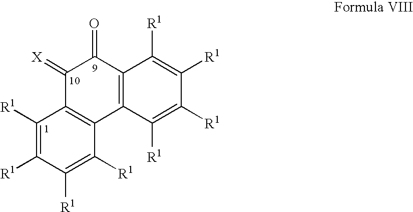
wherein,
R¹ represents H, NO₂, NR⁵R⁶, halogen, cyano, alkyl, alkylaryl, carbonyl, carboxy, COR², or CONR⁵R⁶;
R² and R³ represent independently H, C¹-C⁴ alkyl, aryl, or alkylaryl;
R⁴ represents H, C¹-C⁴ alkyl, aryl, alkylaryl, SO₂—R², NHSO₂R², NHCOR², NHCO₂R², N═CR²R³, or NR⁵R⁶;
R⁵ represents H, C¹-C⁴ alkyl, aryl, alkylaryl, (CH₂)ₙCOXR², (CH₂)ₙXR², (CH₂)ₙCO(CH₂)ₘXR², SO₂R², (CH₂)ₙCO(CH₂)ₙCOXR², or (CH₂)ₙCOR²;
R⁶ represents H, C¹-C⁴ alkyl, aryl, alkylaryl, (CH₂)ₙCOX—R², (CH₂)ₙXR², (CH₂)ₙCO(CH₂)ₘXR², SO₂R², (CH₂)ₙCO(CH₂)ₙCOXR², or (CH₂)ₙCOR²;
m=0-3;
n=0-3; and
X represents CR²R³, O, NR⁴.

The Compound of Formula VIII may be of the formula:

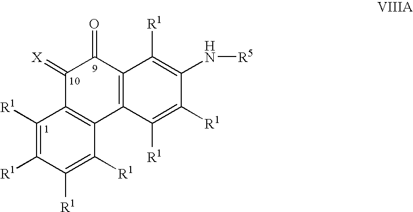

(1) General Synthesis

Phenanthrene-9,10-dione may be nitrated to yield the corresponding 2-nitro phenanthrene-9,10-dione, which upon reduction (H₂, Pd/C, methanol) yields the corresponding 2-amino phenanthrene-9,10-dione. This amine may react with a variety of nucleophiles to yield the aforementioned products [Urbanek et al., (2001), J Med Chem, 44, 11, 1777-93]

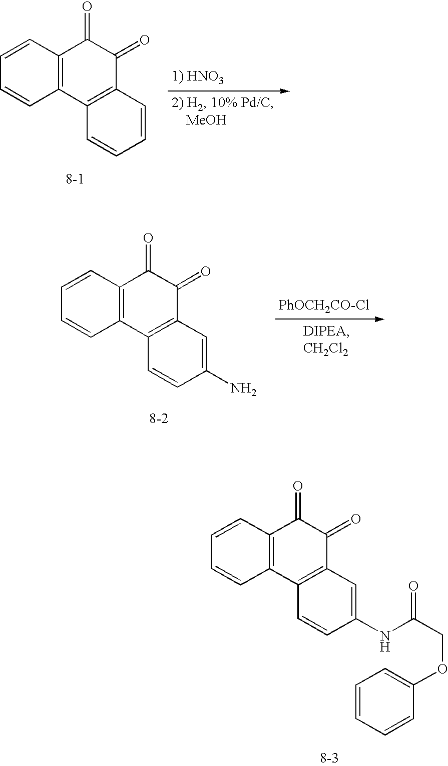

Penanthrene-9,10-dione may react with aromatic or aliphatic amines and upon loss of water yield the corresponding imino ketone compounds. The general synthesis is shown below.

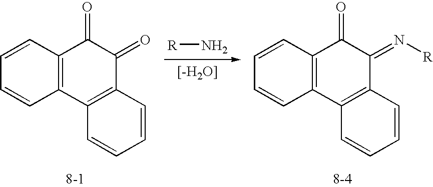

i. Specifications For Isatins

The compound of the present invention may also be an isatin of the formula: 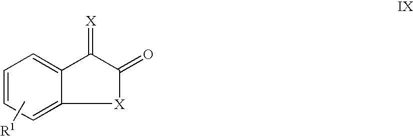
wherein,
R¹ represents H, NO₂, NR⁵R⁶, halogen, cyano, alkyl, alkylaryl, carbonyl, carboxy, COR², CONR⁵R⁶, SO₃R², or SO₂NR²R³;
R² and R³ represent independently H, C¹-C⁴ alkyl, aryl, or alkylaryl;
R⁴ represents H, C¹-C⁴ alkyl, aryl, alkylaryl, SO₂—R², NHSO₂R², NHCOR², NHCO₂R², N═CR²R³, or NR⁵R⁶;
R⁵ represents H, C¹-C⁴ alkyl, aryl, alkylaryl, (CH₂)ₙCOX—R², (CH₂)ₙX—R², (CH₂)ₙCO(CH₂)ₘXR², SO₂R², (CH₂)ₙCO(CH₂)ₙCOXR², or (CH₂)ₙCOR²;
R⁶ represents H, C¹-C⁴ alkyl, aryl, alkylaryl, (CH₂)ₙCOX—R², (CH₂)ₙX—R², (CH₂)ₙCO(CH₂)ₘXR², SO₂R², (CH₂)ₙCO(CH₂)ₙCOXR², or (CH₂)ₙCOR²;
m=0-3;
n=0-3; and
X represents CR²R³, O, NR⁴.

The Compound of Formula IX may be selected from the following:

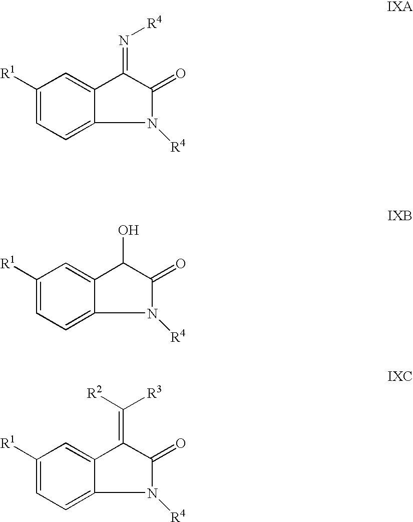

(1) General Synthesis

Compounds of Formula IX may be readily synthesized from suitable substituted aniline. Isatins may be prepared via the classic Sandmeyer reaction which converts substituted anilines with hydroxylamine and chloral hydrate to the intermediate isonitrosoacetanilide, which can then be cyclized in concentrated sulfuric acid. Alternate routes for meta substituted anilines is also known [Bramson et al., (2001), Journal Of Medicinal Chemistry, 44, 25, 4339-4358]. The general synthesis is shown below.

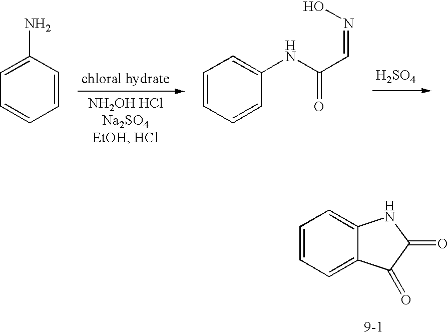

The substituted or unsubstituted isatins, i.e. 9-1, may then be reacted with arylhydrazines in ethanol to yield the corresponding isatin hydrazone (9-2). Alternatively, substituted or unsubstituted isatins may be reduced using classic Wolf Kishner conditions to the corresponding oxindole 9-3. Condensation of substituted or unsubstituted aldehydes or ketones with oxindoles, 9-3, may yield the corresponding enone products, of 9-4. The general synthesis for Formula 9-4 is shown below.

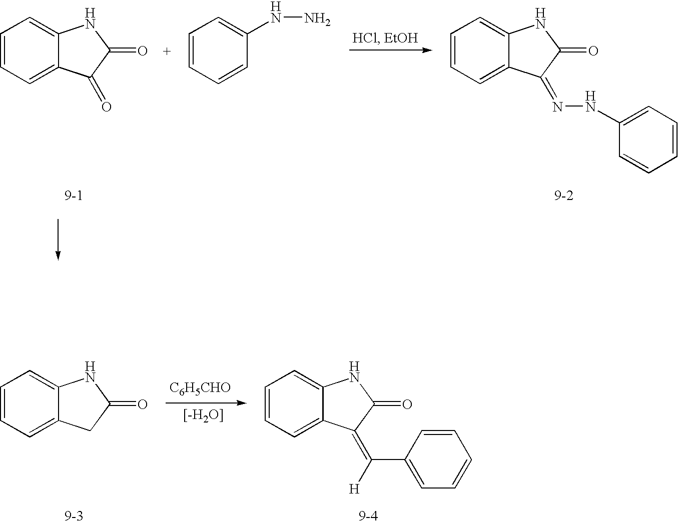

j. Phenanthrols

The compounds of the present invention may also be a substituted phenanthren-9-ols of the formula: 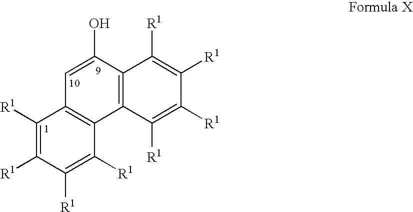
wherein,
R¹ represents H, NO₂, NR⁵R⁶, halogen, cyano, alkyl, alkylaryl, carbonyl, carboxy, COR², or CONR⁵R⁶;
R² and R³ represent independently H, C¹-C⁴ alkyl, aryl, or alkylaryl;
R⁴ represents H, C¹-C⁴ alkyl, aryl, alkylaryl, SO₂—R², NHSO₂R², NHCOR², NHCO₂R², N═CR²R³, or NR⁵R⁶;
R⁵ represents H, C¹-C⁴ alkyl, aryl, alkylaryl, (CH₂)ₙCOXR², (CH₂)ₙX—R², (CH₂)ₙCO(CH₂)ₘXR², SO₂R², (CH₂)ₙCO(CH₂)ₙCOXR², or (CH₂)ₙCOR²;
R⁰ represents H, C¹-C⁴ alkyl, aryl, alkylaryl, (CH₂)ₙCOX—R², (CH₂)ₙX—R², (CH₂)ₙCO(CH₂)ₘXR², SO₂R², (CH₂)ₙCO(CH₂)ₙCOXR², (CH₂)ₙCOR²;
m=0-3;
n=0-3; and
X represents CR²R³, O, NR⁴.

(1) General synthesis

A number of phenanthrols of Formula X may be prepared using the following scheme:

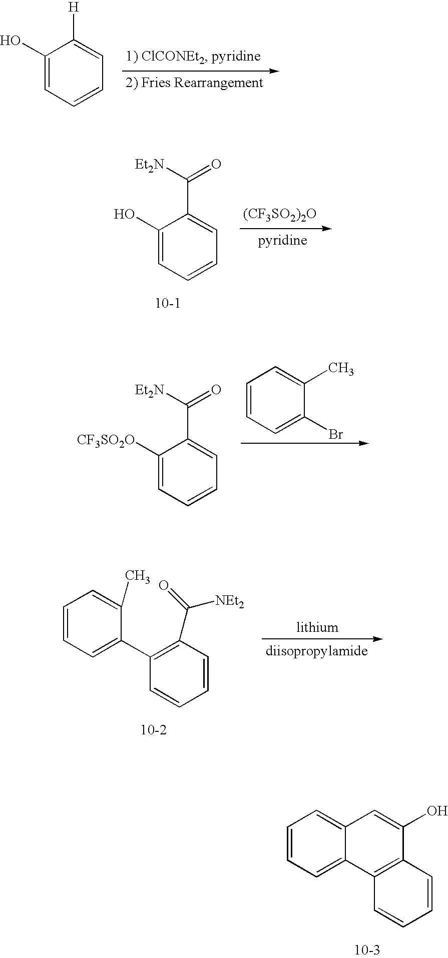

The synthesize of 9-phenanthrols, of general Formula X, may start from commercially available phenols, which may be acylated to give the corresponding carbamates, which may subsequently undergo Fries Rearrangement to the amide 10-1. The amide may be converted to the corresponding triflate which upon treatment with aryl bromides under metal-mediated coupling conditions provide the substituted biphenyl amide 10-2. Methyl H— abstraction with strong base and subsequent attack on the amide may provide the desired substituted phenanthols (10-3) [Cai, X. et al. Can. J. Chem. (2004), 82(2), 195-205; and Fu, J. M; Snieckus, V. Can. J. Chem. (2000), 78(6), 905-919].

An alternate approach starts with readily available substituted biphenyl carboxylic acids [Chatterjea et al., (1979), Indian Journal of Chemistry Section B. Organic Chemistry Including Medicinal Chemistry, 17B, 4, 329-32].

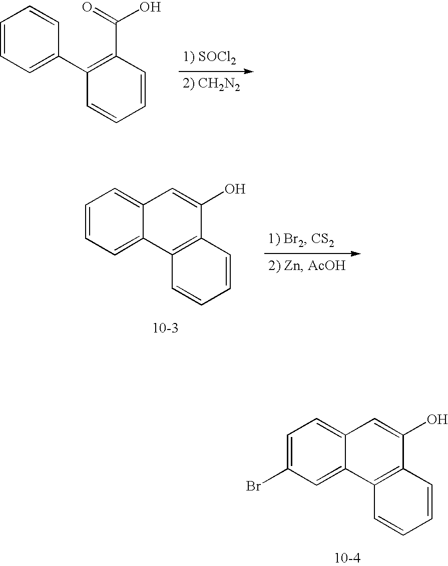

Selective bromination of unsubstituted 9-phenanthrol yielding 9-Bromo-3-phenanthrol (10-4) has also been reported [Ota and Shintani, (1987), Nippon Kagaku Kaishi, 4, 762-4].

k. Naphthalene-1,2 diones

The compound of the present invention may also be a substituted naphthalene-1,2-dione of the formula: 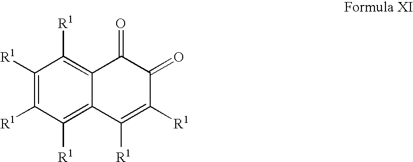
wherein,
R¹ represents Independently chosen from H, NO₂, NR³R⁴, halogen, cyano, alkyl, alkylaryl, carbonyl, carboxy, (CH₂)ₙCOXR³, COR², SO₃—R², SO₂N—R³ R⁴, NHSO₂—R³, NHCO₂R³, NHCOR³, NHCOCO₂R², NR³R⁴, or CONR³R⁴;
R² represents H, C¹-C⁴ alkyl, aryl, or alkylaryl;
R³ and R⁴ represent independently H, C¹-C⁴ alkyl, aryl, alkylaryl, (CH₂)ₙCOXR², (CH₂)ₙCO(CH₂)ₘXR², or (CH₂)ₙOR²;
m=0-3;
n=0-3; and
X represents O, NR².

The Compound of Formula XI may be selected from the following:

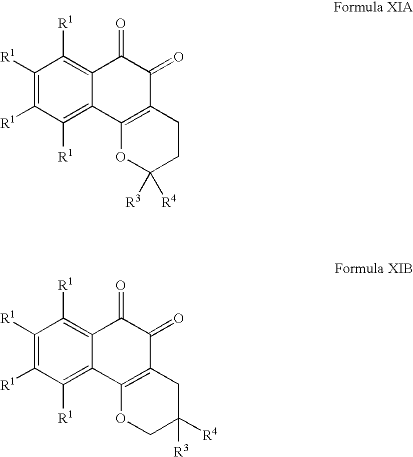

(1) General Synthesis

Compounds of Formula XI may be synthesized using substituted naphthols [Ahn et al., (2002), Bioorganic & Medicinal Chemistry Letters, 12, 15, 1941-1946] using the following scheme.

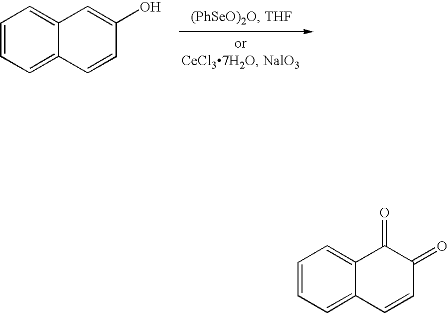

Readily available naphthalene-1,2 diones may also undergo aryl coupling to yield 11-1 [Urbanek et al., (2001), J Med Chem, 44, 11, 1777-93].

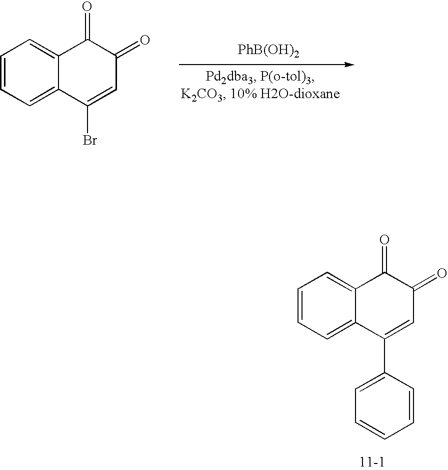

Compounds of Formula XIA (from the rhinacanthone natural product) may be synthesized using methods described in Kongkathip et al., (2003), Bioorganic & Medicinal Chemistry, 11, 14, 3179-3191. Compounds of Formula XIIB may be prepared from substituted 1-hydroxy-2-naphthoic acids [Kongkathip et al., (2003), Bioorganic & Medicinal Chemistry, 11, 14, 3179-3191].

1. Naphthalene-1,4 diones

The compound of the present invention may also be a substituted naphthalene-1,4-dione of the formula: 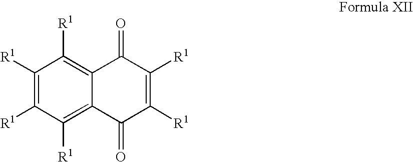
wherein
R¹ represents H, NO₂, NR³R⁴, halogen, cyano, alkyl, alkylaryl, carbonyl, carboxy, (CH₂)ₙCOXR³, COR², SO₃R², SO₂N—R³R⁴, NHSO₂—R³, NHCO₂R³, NHCOR³, NHCOCO₂R², NR³R⁴, or CON—R³R⁴;
R² represents H, C¹-C⁴ alkyl, aryl, or alkylaryl;
R³ and R⁴ represents independently H, C¹-C⁴ alkyl, aryl, alkylaryl, (CH₂)ₙCOXR², (CH₂)ₙCO(CH₂)ₘXR², or (CH₂)ₙOR²;
m=0-3;
n=0-3; and
X represents O, NR².

(1) General Synthesis

Substituted 1-naphthols can be converted to the corresponding napthathene-1,4 dione series using standard known methodology [Kongkathip et al.; (2003), Bioorganic & Medicinal Chemistry, 11, 14, 3179-3191]. Commercially available naphthalene-1,4 diones can also be further modified.

m. Vanadate-Based PTEN Inhibitors

The compound of the present invention may also be a vanadate-based compound selected from the following:

| STRUCTURE | Compound Name | SF Number |
|---|---|---|
| 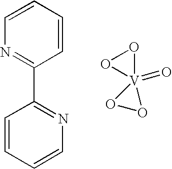 | Potassium Bisperoxo(bipyridine) oxovanadate (V) | SF1668-101 |
| 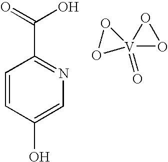 | Dipotassium Bisperoxo (5-hydroxypyridine-2- carboxyl)oxovanadate (V) | SF1674-101 |
| 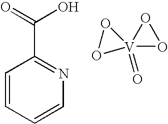 | Dipotassium Bisperoxo (picolinato)oxovanadate (V) | SF1675-101 |
| 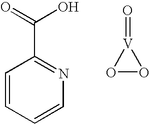 | Monoperoxo(picolinato) oxovanadate(V) | SF1677-000 |
| 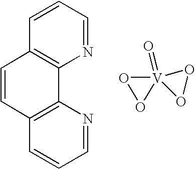 | Potassium Bisperoxo (1,10-phenanthroline) oxovanadate (V) | SF1678-101 |
| 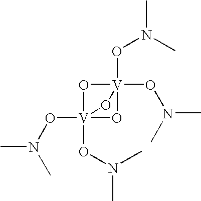 | bis(N,N- Dimethylhydroxamido) hydroxooxovanadate | SF1682-000 |

Some vanadates are competitive reversible inhibitors of protein tyrosine phosphatase (PTPase) [Posner, B. I.;. et al., J Biol Chem 1994, 269, (6), 4596-604] Recently, Woscholski and coworkers have described the use of bisperoxovanadium (bpV) and other vanadate derivatives as inhibitors for PTEN. Interestingly, the bpVs did not display selective inhibition toward PTEN using the assay conditions reported herein [Schmid, A. C.; Byrne, R. D.; Vilar, R.; Woscholski, R., FEBS Lett 2004, 566, (1-3), 35-8.; and Schmid, A. C.; Woscholski, R., Biochem Soc Trans 2004, 32, (Pt 2), 348-9].

n. T1-Loop Binding Elements

The compound of the present invention may also comprise structural elements that allow the compound to physically fit into the catalytic dephosphorylation binding pocket of PTEN. From the crystal structure of PTEN (Jie-Oh Lee et al., Cell, 99:323-334, 1999) it is apparent that PTEN's catalytic binding pocket is larger in width than the binding pocket of other phosphatases such as PTP1B and VHR. From an inspection of the crystal structure the extra width to PTEN's binding pocket is due to the T1loop (Jie-Oh Lee et al. Cell. 99:323-334, 1999). Therefore, compounds that comprise such structural elements may be capable of inhibiting PTEN by binding in the phosphatase catalytic site and which also occupy the space made available by the presence of PTEN's T1 loop.

Because of the high positive charge that is expected to exist in the T1 loop (due to PTEN's ability to accommodate the highly negative charged phosphate groups) at positions 4 and 5 (especially 5) of the inositol ring group of PTEN's substrate (inositol-3,4,5-triphosphate), the compound of the present invention may contain a group that exists at physiological pH in significantly anionic form, such as at least 5% of the molecular species at pH of 7.4 are anionic charged. The anionic groups may be capable of binding to PTEN in the T1 loop of the peptide structure in solution. Representative examples of such groups may be selected from the following: 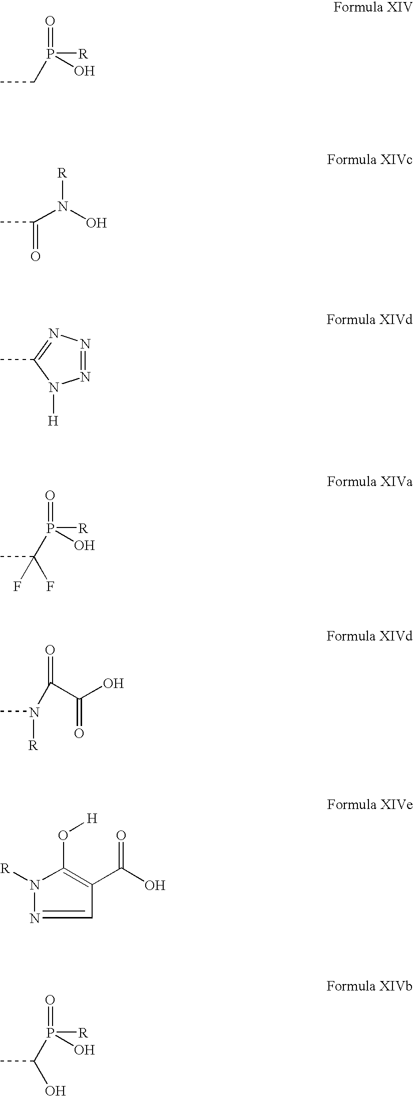
wherein, R is independently chosen from H, OH, O-alkyl, alkyl, SH, S-alkyl, NH₂, NH-alkyl, N-(alkyl)₂ where alkyl is a small, C1-C4 alkyl moiety. The dashed lines represent the connection to the formulas of the compounds of the invention described for Formulas I through XIII above. The groups may be further evaluated in silico for their ability to fill the T1 loop space by standard molecular docking procedures.

Such T1-loop binding groups may be incorporated into compounds of Formula I-XIII. Incorporation of the groups may impart selectivity of the molecules to inhibition of PTEN. Preparation of groups XIVa-XIVd are well established in the literature. Compounds of Formula XIVe may be prepared by methods disclosed in Wilson et al., Bioorganic & Medicinal Chemistry Letters, vol 6, No. 9, pp1043-1046, 1996. Incorporation of these groups into the Formulas I-XIII of the invention is by standard synthetic methods easily attainable by those skilled in the art. Examples of such incorporation by simply utilizing appropriate starting materials is illustrated by the conversion of 7-9 to one incorporating the above groups:

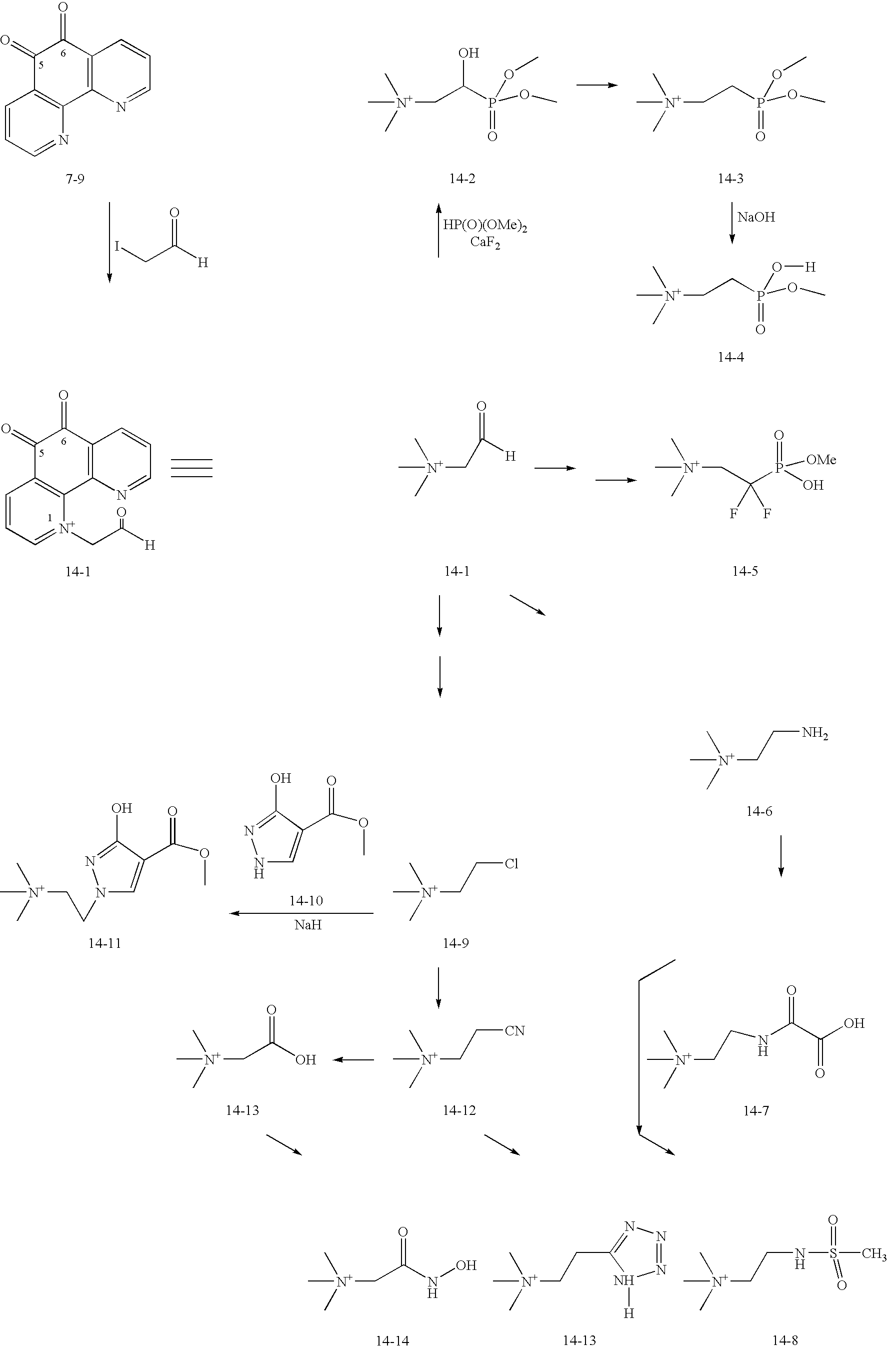

For example, compound 14-1 may be prepared by quaternization under forcing conditions from commercially available dione 7-9 as shown. The iodoacetaldehyde shown can also be a masked as an equivalent such as the dimethyl or diethyl acetal or made in situ from halogen exchange (NaI/actonitrile) with the more stable commercially available chloroacetaldehyde or chloroacetaldehyde dimethyl or diethyl acetal. The conversion of aldehydes to numerous phosphorous acid species (including aminophosphonic acids) is well established in the literature including reduction of the hydroxyl group and cleavage under basic conditions to the mono-phosphonic acid 14-4. Likewise literature exists demonstrating the conversion of aldehydes to mono and di-fluoro-phosphonic acids and their esters which can act as phosphate mimics (MS Smyth et al; “A General Method for the Preparation of Benzylic α,α-Difluorophosphonic Acids: Non-Hydrolyzable Mimetics of Phosphotyrosine”; Tetrahedron Letters, vol 33, No. 29, pp 4137-4140, 1992). Additionally, intermediate aldehyde 14-1 can be converted to a chloro species 14-9 under a variety of conditions (reduction followed by phosphorous trichloride) to yield 14-9 from which reaction with nucleophiles obtained from 14-10 molecules with sodium hydride (Wilson et al “Bone Targeted Drugs 2. Identification of Heterocycles with Hydroxyapatite Affinity”, Bioroganic & Medicinal Chemistry Letters, vol 6, No. 9, pp 1047-1050, 1996) to give compounds like 14-11 from which the methyl ester can be cleaved to yield a phosphate mimicking group. Also 14-9 can be converted to the cyano group (KCN nucleophilic displacement) and then converted with sodium azide and zinc salts to the lipophilic phosphate mimic group shown by compound 14-13 (ZP Demko, KB Sharpless “Preparation of 5-Substituted 1H-Tetrazoles from nitrites in water”; J. Org. Chem. 2001, 66(24), pp 7945-50). Additionally 14-1 can be reductively aminated to 14-6 and acylated by an oxalyl mono equivalent to give oxamic acids like 14-7 which are also phosphate mimics or alternatively sufonylated to give sulfonamides such as 14-8. Lastly, intermediate cyano 14-12 can be hydrolyzed to the acid 14-13 under strongly acidic conditions and the acid can then be converted by standard coupling conditions with hydroxyl amine to give hydroxamic acids such as 14-14.

3. Salts

The compounds of the present invention are useful in various pharmaceutically acceptable salt forms. The term “pharmaceutically acceptable salt” refers to those salt forms which would be apparent to the pharmaceutical chemist, i.e., those which are substantially non-toxic and which provide the desired pharmacokinetic properties, palatability, absorption, distribution, metabolism or excretion. Other factors, more practical in nature, which are also important in the selection, are cost of the raw materials, ease of crystallization, yield, stability, hygroscopicity and flowability of the resulting bulk drug. Conveniently, pharmaceutical compositions may be prepared from the active ingredients or their pharmaceutically acceptable salts in combination with pharmaceutically acceptable carriers.

Pharmaceutically acceptable salts of the compounds, which are suitable for use in the methods and compositions of the present invention include, but are not limited to, salts formed with a variety of organic and inorganic acids such as hydrogen chloride, hydroxymethane sulfonic acid, hydrogen bromide, methanesulfonic acid, sulfuric acid, acetic acid, trifluoroacetic acid, maleic acid, benzenesulfonic acid, toluenesulfonic acid, sulfamic acid, glycolic acid, stearic acid, lactic acid, malic acid, pamoic acid, sulfanilic acid, 2-acetoxybenzoic acid, fumaric acid, toluenesulfonic acid, methanesulfonic acid, ethanedisulfonic acid, oxalic acid, isethonic acid, and include various other pharmaceutically acceptable salts, such as, e.g., nitrates, phosphates, borates, tartrates, citrates, succinates, benzoates, ascorbates, salicylates, and the like. Cations such as quaternary ammonium ions are contemplated as pharmaceutically acceptable counterions for anionic moieties.

The salts of the compounds of the present invention include hydrochloride salts, methanesulfonic acid salts and trifluoroacetic acid salts with methanesulfonic acid salts being more preferred. In addition, Pharmaceutically acceptable salts of the compounds may be formed with alkali metals such as sodium, potassium and lithium; alkaline earth metals such as calcium and magnesium; organic bases such as dicyclohexylamine, tributylamine, and pyridine; and amino acids such as arginine, lysine and the like.

The pharmaceutically acceptable salts may be synthesized by conventional chemical methods. Generally, the salts are prepared by reacting the free base or acid with stoichiometric amounts or with an excess of the desired salt-forming inorganic or organic acid or base, in a suitable solvent or solvent combination.

In general, the counterions of the salts of the compounds are determined by the reactants used to synthesized the compounds. There may be a mixture of counterions of the salts, depending on the reactants. For example, where NaI is added to facilitate the reaction the counterion may be a mixture of Cl and I counter anions. Furthermore preparatory HPLC may cause the original counterion to be exchanged by acetate anions when acetic acid is present in the eluent. The counterions of the salts may be exchanged to a different counterion. The counterions are preferably exchanged for a pharmaceutically acceptable counterion to form the salts described above. Procedures for exchanging counterions are described in WO 2002/042265, WO 2002/042276 and S. D. Clas, “Quaterized Colestipol, an improved bile salt adsorbent: In Vitro studies.” Journal of Pharmaceutical Sciences, 80(2): 128-131 (1991), the contents of which are incorporated herein by reference. For clarity reasons the counterions are not explicitly shown in the chemical structures herein. Additionally, common charged groups such as carboxylic acids and phosphonic acids may be utilized as their ester counterparts in a prodrug fashion wherein the ester is cleaved in vivo to generate the active PTEN inhibitor.

4. Composition

The present invention is also related to composition comprising one or more compounds of the present invention. The compositions may further comprise one or more pharmaceutically acceptable additional ingredient(s) such as alum, stabilizers, antimicrobial agents, buffers, coloring agents, flavoring agents, adjuvants, and the like.

a. Formulation

The compositions may be in the form of tablets or lozenges formulated in a conventional manner. For example, tablets and capsules for oral administration may contain conventional excipients including, but not limited to, binding agents, fillers, lubricants, disintegrants and wetting agents. Binding agents include, but are not limited to, syrup, accacia, gelatin, sorbitol, tragacanth, mucilage of starch and polyvinylpyrrolidone. Fillers include, but are not limited to, lactose, sugar, microcrystalline cellulose, maizestarch, calcium phosphate, and sorbitol. Lubricants include, but are not limited to, magnesium stearate, stearic acid, talc, polyethylene glycol, and silica. Disintegrants include, but are not limited to, potato starch and sodium starch glycollate. Wetting agents include, but are not limited to, sodium lauryl sulfate). Tablets may be coated according to methods well known in the art.

The compositions may also be liquid formulations including, but not limited to, aqueous or oily suspensions, solutions, emulsions, syrups, and elixirs. The compositions may also be formulated as a dry product for constitution with water or other suitable vehicle before use. Such liquid preparations may contain additives including, but not limited to, suspending agents, emulsifying agents, nonaqueous vehicles and preservatives. Suspending agent include, but are not limited to, sorbitol syrup, methyl cellulose, glucose/sugar syrup, gelatin, hydroxyethylcellulose, carboxymethyl cellulose, aluminum stearate gel, and hydrogenated edible fats. Emulsifying agents include, but are not limited to, lecithin, sorbitan monooleate, and acacia. Nonaqueous vehicles include, but are not limited to, edible oils, almond oil, fractionated coconut oil, oily esters, propylene glycol, and ethyl alcohol. Preservatives include, but are not limited to, methyl or propyl p-hydroxybenzoate and sorbic acid.

The compositions may also be formulated as suppositories, which may contain suppository bases including, but not limited to, cocoa butter or glycerides. Compositions of the present invention may also be formulated for inhalation, which may be in a form including, but not limited to, a solution, suspension, or emulsion that may be administered as a dry powder or in the form of an aerosol using a propellant, such as dichlorodifluoromethane or trichlorofluoromethane. Compositions of the present invention may also be formulated transdermal formulations comprising aqueous or nonaqueous vehicles including, but not limited to, creams, ointments, lotions, pastes, medicated plaster, patch, or membrane.

The compositions may also be formulated for parenteral administration including, but not limited to, by injection or continuous infusion. Formulations for injection may be in the form of suspensions, solutions, or emulsions in oily or aqueous vehicles, and may contain formulation agents including, but not limited to, suspending, stabilizing, and dispersing agents. The composition may also be provided in a powder form for reconstitution with a suitable vehicle including, but not limited to, sterile, pyrogen-free water.

The compositions may also be formulated as a depot preparation, which may be administered by implantation or by intramuscular injection. The compositions may be formulated with suitable polymeric or hydrophobic materials (as an emulsion in an acceptable oil, for example), ion exchange resins, or as sparingly soluble derivatives (as a sparingly soluble salt, for example).

The compositions may also be formulated as a liposome preparation. The liposome preparation can comprise liposomes which penetrate the cells of interest or the stratum corneum, and fuse with the cell membrane, resulting in delivery of the contents of the liposome into the cell. For example, liposomes such as those described in U.S. Pat. No. 5,077,211 of Yarosh, U.S. Pat. No. 4,621,023 of Redziniak et al or U.S. Pat. No. 4,508,703 of Redziniak et al. can be used. Compositions intended to target skin conditions may be administered before, during, or after exposure of the skin of the mammal to UV or agents causing oxidative damage. Other suitable formulations may employ niosomes. Niosomes are lipid vesicles similar to liposomes, with membranes consisting largely of non-ionic lipids, some forms of which are effective for transporting compounds across the stratum corneum.

The compositions may be administered in any manner including, but not limited to, orally, parenterally, sublingually, transdermally, rectally, transmucosally, topically, via inhalation, via buccal administration, or combinations thereof. Parenteral administration includes, but is not limited to, intravenous, intraarterial, intraperitoneal, subcutaneous, intramuscular, intrathecal, and intraarticular. The compound may be administered simultaneously or metronomically with other therapeutics. The term “simultaneous” or “simultaneously” as used herein, means that the other therapeutic and the compound of the present invention are administered within 48 hours, preferably 24 hours, more preferably 12 hours, yet more preferably 6 hours, and most preferably 3 hours or less, of each other. The term “metronomically” as used herein means the administration of the compound of the present invention at times different from the other therapeutic and at certain frequency relative to repeat administration of the other therapeutic.

5. Treatment

The present invention is also related to a method of treating a patient suffering from a condition associated with PTEN activity. The PTEN activity may be normal, abnormal, excessive, or constitutively active. By inhibiting PTEN activity, activities such as angiogenesis may be promoted.

The PTEN activity may also be induced by stress. By inhibiting PTEN activity, normal cells such as cardiac myocytes, neuronal cells and bone marrow cells may be protected from apoptosis attributable to cellular stress. The cellular stress may be caused, for example, by hyperthermia, hypoxia or medical treatments such as cancer treatment, open heart surgery, surgery in general, invasive cardiovascular procedures, and general anesthesia. The PTEN inhibitor may be administered before, during, after or a combination thereof with respect to the procedure. Once the cells have been protected or repaired, PTEN activity may be restored to normal levels by stopping administration of the PTEN inhibitor.

The present invention is also related to treating a patient suffering from a heart attack. A PTEN inhibitor may be administered to a patient suffering from a heart attack or at risk of suffering a heart attack. By suppressing PTEN activity, stressed heart cells may be prevented from committing apoptosis including heart cells suffering from hypoxia. Moreover, reduced PTEN activity may promoted new blood vessels to grow in vivo including for example the diseased or damaged heart.

The present invention is also related to treating a patient undergoing radiation- or chemotherapy treatment. A PTEN inhibitor may be administered to a patient undergoing cancer treatment. By suppressing PTEN activity, sensitive tissues such as the hematopoietic system (including immune system), the epithelium of the gut, and hair follicles may be protected from undergoing apoptosis. A PTEN inhibitor may be administered to protect animals and humans such as military personnel or civilians that are exposed to ionizing radiation or chemical poisoning from accidents or terrorist activities. When treating cancer, the compounds of the present invention may be administered in combination with a chemotherapy treatment, such as a cytotoxic agent, cytostatic agent, or combination thereof. Cytotoxic agents prevent cancer cells from multiplying by: (1) interfering with the cell's ability to replicate DNA and (2) inducing cell death and/or apoptosis in the cancer cells. Cytostatic agents act via modulating, interfering or inhibiting the processes of cellular signal transduction which regulate cell proliferation and sometimes at low continuous levels.

Classes of compounds that may be used as cytotoxic agents include the following: alkylating agents (including, without limitation, nitrogen mustards, ethylenimine derivatives, alkyl sulfonates, nitrosoureas and triazenes): uracil mustard, chlormethine, cyclophosphamide (Cytoxan®), ifosfamide, melphalan, chlorambucil, pipobroman, triethylene-melamine, triethylenethiophosphoramine, busulfan, carmustine, lomustine, streptozocin, dacarbazine, and temozolomide; antimetabolites (including, without limitation, folic acid antagonists, pyrimidine analogs, purine analogs and adenosine deaminase inhibitors): methotrexate, 5-fluorouracil, floxuridine, cytarabine, 6-mercaptopurine, 6-thioguanine, fludarabine phosphate, pentostatine, and gemcitabine; natural products and their derivatives (for example, vinca alkaloids, antitumor antibiotics, enzymes, lymphokines and epipodophyllotoxins): vinblastine, vincristine, vindesine, bleomycin, dactinomycin, daunorubicin, doxorubicin, epirubicin, idarubicin, ara-c, paclitaxel (paclitaxel is commercially available as Taxol®), mithramycin, deoxyco-formycin, mitomycin-c, 1-asparaginase, interferons (preferably IFN-α), etoposide, and teniposide.

Other proliferative cytotoxic agents are navelbene, CPT-11, anastrazole, letrazole, capecitabine, reloxafine, cyclophosphamide, ifosamide, and droloxafine.

Microtubule affecting agents interfere with cellular mitosis and are well known in the art for their cytotoxic activity. Microtubule affecting agents useful in the invention include, but are not limited to, allocolchicine (NSC 406042), halichondrin B (NSC 609395), colchicine (NSC 757), colchicine derivatives (e.g., NSC 33410), dolastatin 10 (NSC 376128), maytansine (NSC 153858), rhizoxin (NSC 332598), paclitaxel (Taxol®, NSC 125973), Taxol® derivatives (e.g., derivatives (e.g., NSC 608832), thiocolchicine NSC 361792), trityl cysteine (NSC 83265), vinblastine sulfate (NSC 49842), vincristine sulfate (NSC 67574), natural and synthetic epothilones including but not limited to epothilone A, epothilone B, and discodermolide (see Service, (1996) Science, 274:2009) estramustine, nocodazole, MAP4, and the like. Examples of such agents are also described in Bulinski (1997) J. Cell Sci. 110:3055 3064; Panda (1997) Proc. Natl. Acad. Sci. USA 94:10560-10564; Muhlradt (1997) Cancer Res. 57:3344-3346; Nicolaou (1997) Nature 387:268-272; Vasquez (1997) Mol. Biol. Cell. 8:973-985; and Panda (1996) J. Biol. Chem 271:29807-29812.

Also suitable are cytotoxic agents such as epidophyllotoxin; an antineoplastic enzyme; a topoisomerase inhibitor; procarbazine; mitoxantrone; platinum coordination complexes such as cis-platin and carboplatin; biological response modifiers; growth inhibitors; antihormonal therapeutic agents; leucovorin; tegafur; and haematopoietic growth factors.

Cytostatic agents that may be used include, but are not limited to, hormones and steroids (including synthetic analogs): 17-alpha-ethinylestradiol, diethylstilbestrol, testosterone, prednisone, fluoxymesterone, dromostanolone propionate, testolactone, megestrolacetate, methylprednisolone, methyl-testosterone, prednisolone, triamcinolone, hlorotrianisene, hydroxyprogesterone, aminoglutethimide, estramustine, medroxyprogesteroneacetate, leuprolide, flutamide, toremifene, zoladex.

Other cytostatic agents are antiangiogenics such as matrix metalloproteinase inhibitors, and other VEGF inhibitors, such as anti-VEGF antibodies and small molecules such as ZD6474 and SU6668 are also included. Anti-Her2 antibodies from Genetech may also be utilized. A suitable EGFR inhibitor is EKB-569 (an irreversible inhibitor). Also included are Imclone antibody C225 immunospecific for the EGFR, and src inhibitors.

Also suitable for use as an cytostatic agent is Casodex® (bicalutamide, Astra Zeneca) which renders androgen-dependent carcinomas non-proliferative. Yet another example of a cytostatic agent is the antiestrogen Tamoxifen® which inhibits the proliferation or growth of estrogen dependent breast cancer. Inhibitors of the transduction of cellular proliferative signals are cytostatic agents. Representative examples include epidermal growth factor inhibitors, Her-2 inhibitors, MEK-1 kinase inhibitors, MAPK kinase inhibitors, PI3 kinase inhibitors, Src kinase inhibitors, and PDGF inhibitors.

A variety of cancers may be treated according to the present invention including, but not limited to, the following: carcinoma including that of the bladder (including accelerated and metastatic bladder cancer), breast, colon (including colorectal cancer), kidney, liver, lung (including small and non-small cell lung cancer and lung adenocarcinoma), ovary, prostate, testes, genitourinary tract, lymphatic system, rectum, larynx, pancreas (including exocrine pancreatic carcinoma), esophagus, stomach, gall bladder, cervix, thyroid, and skin (including squamous cell carcinoma); hematopoietic tumors of lymphoid lineage including leukemia, acute lymphocytic leukemia, acute lymphoblastic leukemia, B-cell lymphoma, T-cell lymphoma, Hodgkins lymphoma, non-Hodgkins lymphoma, hairy cell lymphoma, histiocytic lymphoma, and Burketts lymphoma; hematopoietic tumors of myeloid lineage including acute and chronic myelogenous leukemias, myelodysplastic syndrome, myeloid leukemia, and promyelocytic leukemia; tumors of the central and peripheral nervous system including astrocytoma, neuroblastoma, glioma, and schwannomas; tumors of mesenchymal origin including fibrosarcoma, rhabdomyoscarcoma, and osteosarcoma; and other tumors including melanoma, xenoderma pigmentosum, keratoactanthoma, seminoma, thyroid follicular cancer, and teratocarcinoma.

The present invention is also related to the use of PTEN inhibitors to enhance the sensitivity of cancer cells to inhibitors of the PI3 kinase pathway. PTEN inhibitors may be administered for a period of time sufficient to make the cancer cells more dependant on PI3 kinase-mediated signals including, but not limited to, downstream signals such as p-AKT and mTOR. Once administration of the PTEN inhibitor is discontinued, the cancer cells may experience a disruption or alteration in the PI3 kinase pathway. The disruption of the PI3 kinase pathway may be anywhere along the pathway including upstream growth factor receptors. While not being bound by theory, the cancer cells may not able to adjust quickly enough and succumb to resulting pro-death signal conditions or at least disruptions in the pro-survival signal conditions.

The present invention is also related to the use of PTEN inhibitors to stimulate cancer “stem cells” to enter into a state whereby they are more susceptible to approved treatments and treatments currently under development such as using a PI3 kinase pathway inhibitor. Cancer stem cells are believed to be the reason that cancer is resistant to treatment because they are quiescent and thus resistant to chemo and radiation therapy. For further discussion on cancer stem cells, see Dean, M., Fojo, T., and Bates, S. “Tumour Stem Cells and Drug Resistance”; Nature Reviews: Cancer, volume 5, April 2005 p 276-284.

The present invention is also related to regenerating or enhancing EPO activity in regenerating neurons. As discussed in Mucke HAM, Neuroprotection and Neuroregeneration—Annual Global Conference, Innsbruck, Austria, Investigational Drug Database MEETING REPORT, Mar. 7-9, 2005, Thomson Scientific, the PI3K/Akt pathway is involved in anti-apoptotic and regeneration-enhancing EPO actions. PTEN. The present invention enables patients to be treated with a small molecule to inhibit PTEN for augmenting immunity, preventing apoptosis in cerebrovaxcular insult and gram negative sepsis, and inhibiting cellular senescence (US Patent Application Publication US 2002/0150954, which is incorporated herein by reference).

The present invention has multiple aspects, illustrated by the following non-limiting examples.

### EXAMPLE 1

### General HPLC Method A

HPLC analysis was performed on a Shimadzu LCMS-2010 and employed a flow rate of 3 ml/min and a starting B concentration of 5%. The B solvent was linearly ramped to 95% concentration at 5.0 minutes, held at 95% until 6.0 minutes, then linearly ramped back down to 5% at 6.5 minutes, where it remained until the end of the run at 7.5 minutes. In addition to mass detection the LC detection consisted of 3 channels: UV absorbance at 254 nm, UV absorbance at 214 nm, and evaporative light scattering (Alltech ELSD 2000). The evaporative light scattering detector was run at 50° C. with a nitrogen flow of 1.5 liters per minute. The CDL and block temperatures of the Shimadzu LCMS-2010 were both 300° C., and the nitrogen nebulizer gas flow was 4.5 L/min. Positive and negative mass spectra were detected from 50 to 2000 m/z. The column was a YMC CombiScreen ODS-AQ, S-5μ particle size, 50 mm long with a 4.6 mm I.D. Mobile phase A was made using HPLC grade B&J water with 0.1% (v/v) HOAc added and mobile phase B was HPLC grade B&J acetonitrile with 0.1% (v/v) HOAc added. This system gives a retention time of 1.50 to 1.60 minutes for a standard commercially available material (4-hydroxyphenylacetic acid; Aldrich Catalog H5000-4; m.p. 149-151° C.) used as a reference standard.

### EXAMPLE 2

### General Preparative HPLC Method

Gradient Preparative HPLC was performed on a Shimadzu system composed of two LC-8A pumps connected to a SIL-10A autosampler and eluting over a reverse phase column (YMC, cat CCAQSOSO52OWT; ODS-AQ CombiPrep, 20 mm×50 mm) and then passing through an MRA variable volume splitter; the smaller stream was then made up to 3 mL/minute using a LC-10ADVP make-up pump (MeOH) and the eluent passed through a variable two channel wavelength UV detector and then split roughly 6:1 to an evaporative light scattering detector (run at 50° C. with a nitrogen flow of 1.5 liters per minute) and a Shimadzu 2010 Mass detector; the larger stream from the MRA splitter then flowed to a Gilson 215 liquid handler serving as a fraction collector triggered by mass, UV absorbance, or ELS peak size.

Different gradients were run starting with the more aqueous solvent A and ramping up to various concentrations of B. Mobile phase A was made using HPLC grade B&J water with 0.1% (v/v) HOAc added and mobile phase B was HPLC grade B&J acetonitrile with 0.1% (v/v) HOAc added.

### EXAMPLE 3

### PTEN Inhibition Assay: General Screening

PTEN protein is a phosphatase capable of dephosphorylating the D-3 position of inositol phospholipids, such as phosphatidyl inositol 3,4,5-triphosphate, and removing the phosphate group on the residue of poly glutamic-tyrosine peptide (EEEEYp)n. Free phosphate, which is a product of the PTEN dephosphorylation reaction, can be detected by colorimetric reaction with commercially available malachite green solutions (Upstate). PTEN inhibitors were evaluated in an inhibition assay conducted in half-volume 96 well plates in 25 ul total volume per well containing 2 mM dithiothreitol (DTT) and 0.1 mM Tris buffer, pH 8.0 and up to 3 ug total protein of PTEN. Small volumes of the test inhibitor candidates (stock concentrated solutions of 25 mM in DMSO) were mixed with the PTEN solution at room temperature for about 10 minutes and then substrate was added. The reaction mix was then incubated in 37° C. for 20 minutes. Subsequent to this a 100 ul aliquot of malachite green buffer (Upstate, Charlottesville, Va.) was added to develop the color in the dark at room temperature (this solution also stopped the dephosphorylation reaction). A SpectraMax Plus spectrophotometric plate reader (Molecular Devices, Sunnydale, Calif.) was used to measure the optical density at 650 nanometers. The initial screening concentration of inhibitor candidates was 250 uM and candidates with inhibition greater than 50% compared with a no-inhibitor control group were then evaluated further to determine IC50 values. The PTEN protein can also be prepared by literature methods [i.e. from cell extracts of bacteria expressing genetic reconstituted Glutathione-S-transferase (GST)-PTEN fusion protein whereupon the GST-PTEN in the cell extract is bound onto and purified from Glutathione Sepharose 4B gel (Amersham, Piscataway, N.J.)]. PTEN is also available from commercial sources.

The PTEN reaction substate PIP3 Phospholipid vesicle (PLV) was utilized at about 50 uM in the final reaction mixture (based on component concentration). The PLV was made based on literature methods (Upstate product manual: www.upstate.com/img/coa; Maehama T, Taylor GS, Slama J T and Dixon J E; 2000, Analytical Biochemistry 279, 248-250). The PLV was prepared from 1,2Dipalmitoyl-sn-glycero-3-phospho-1-D-myo-inositol-3,4,5-tris phosphate (Biomol, Plymouth Meeting, Pa.) by sonication in the presence of Synthetic Phospholipid Blend DOPC/DOPS (Avantilipids, Alabaster, Ala.). Another PTEN reaction substrate, water soluble PIP3 Echelon Biosciences, Salt Lake City, Utah, was utilized at a working concentration of 100 uM. A third PTEN substrate used was a phosphorylated poly glutamic-tyrosine peptide designated (EEEEYp)ⁿ where n=2 or 3 (Biofacilities of Indiana University, Indianapolis, Ind.). The working concentration of the phosphorylated tyrosine substrate was 200 uM. When the assay was run on multiple occasions and gave slightly different percent inhibitions those are reported as a range of inhibition found.

### EXAMPLE 4

### PTEN Inhibition Assay: IC50 Determinations

To determine the dose response of potential PTEN inhibitors, doses of test compounds ranging from 1 nM to 250 uM (final reaction mix concentrations) were evaluated in the general PTEN inhibition assay. To obtain performed IC50 data, two separate rounds of the dose response assay were performed. In the first round, PTEN activity was tested in the presence of inhibitor at 10 fold serial dilutions ranging from 1 nM to 250 uM. Once the concentration range was determined, at which PTEN activity changes dramatically, two additional concentration data points within this range were added and the PTEN inhibition assay was then rerun for the second round. The PTEN inhibition IC50 is presented as the inhibitor concentration at which 50% of the PTEN activity (measured by phosphate production and compared to un-inhibited control samples) was found. The IC50 determination from the data was made using Prism software (GraphPad Software, San Diego, Calif.). When the assay was run on multiple occasions and gave slightly different IC50 then those are reported as a range of IC50 found.

### EXAMPLE 5

### PTP1B General Inhibition Assay

Protein Tyrosine Phosphatase 1B (PTP1B) dephosphorylates polypeptide (EEEEYp)ⁿ and p-nitrophenyl phosphate (pNPP). Free phosphate, which was a product of the PTP1B dephosphorylation reaction, was detected by malachite (detected by colorimetric reaction with commercially available malachite green solutions (Upstate)). The PTP1B inhibition assay was conducted in half-volume 96 well plates in 25 ul total volume containing 2 mM dithiothreitol (DTT) and 0.1 mM Tris buffer, pH 8.0. The inhibitor candidates were allowed to react with PTP1B (Upstate; Charlottesville, Va.) in room temperature for 10 minutes, substrate was then added, and the reaction mix was incubated at 37° C. for 20 minutes. A 100 ul portion of malachite green buffer solution (Upstate, Charlottesville, Va.) was added to develop the color in the dark at room temperature. A SpectraMax Plus spectrophotometric plate reader (Molecular Devices, Sunnydale, Calif.) was then used to measure the optical density at 650 nanometers. Potential PTP1B inhibitors screening was based on this PTP1B inhibition assay with the inhibitor concentration set at 250 uM. Compounds showing greater than 50% compared with no inhibitor control were then evaluated further for dose response and IC50 determination. Using the reaction substrate poly glutamic tyrosine polypeptide (EEEEYp)ⁿ (where n=2 or 3) (Biofacilities of Indiana University, Indianapolis, Ind.) or pNPP, (Aldrich, Milwaukee, Wis.).

### EXAMPLE 6

### PTP1B Inhibition Assay: IC50 Determinations

To determine the dose response of potential PTP1B inhibitors, doses of test compounds ranging from 1 nM to 250 uM (final reaction mix concentrations) were evaluated in the general PTP1B inhibition assay. To obtain accurate IC50 data, two separate rounds of the dose response assay were performed. In the first round, PTP1B activity was tested in the presence of an inhibitor at 10 fold serial dilutions ranging from 1 nM to 250 uM. Once the concentration range at which PTEN activity changes dramatically, two additional concentration data points within this range were added and the PTP1B inhibition assay was then rerun for the second round. The PTP1B inhibition IC50 is presented as the inhibitor concentration at which 50% of the PTP1B activity (measured by phosphate production and compared to un-inhibited control samples) was found. The IC50 determination from the data was made using Prism software (GraphPad Software, San Diego, Calif.).

### EXAMPLE 7

### General MTT Cytotoxicity Assay

MTT (dimethylthiazol-diphenyl-tetrazolium bromide, Aldrich, Milwaukee, Wis.) uptake was used to evaluate the toxic effect of PTEN inhibitor candidates on Human Brain Endothelial Cells (HBEC), Mouse Embryo Fibroblast (MEF) and Mouse Fibroblast NIH3T3 cells. The assay was conducted in 96 well plates. One day prior to treating the cells, 5000 cells were seeded into each well in complete serum medium (10% Fetal Bovine Serum) or under serum starvation conditions (1% Fetal Bovine Serum medium). Fetal Bovine Serum (FBS) was obtained from Invitrogen, Carlsbad, Calif.). Plates were then incubated overnight at 37° C. with a 5% CO₂ atmosphere. The cells were then treated with the test PTEN inhibitors at doses ranging from 1 pM to 1 mM (test solutions were in complete serum complete or serum starvation medium). The cells were then incubated for 24 hours at 37° C. at 5% CO₂. The media was then aspirated and the adherent cells stained by the addition of 200 ul/well of 0.5 mg/ml MTT for 4 hours at 37° C. The MTT solution was then aspirated and each well was treated with 150 uL/well of dimethylsulfoxide (DMSO) to dissolve the cell-associated MTT stain. A SpectraMax Plus spectrophotometric plate reader (Molecular Devices, Sunnydale, Calif.) was used to measure the optical density of each well at 570 nanometers. The IC50 for each inhibitor is presented as the concentration at which 50% of the highest optical density (OD570 nm) was observed representing 50% of the cells being viable. The IC50 determination from the data was made using Prism software (GraphPad Software, San Diego, Calif.).

### EXAMPLE 8

### MTT Assay for Determining Toxicity

Cytotoxicity of the potential PTEN inhibitors SF1720, SF1773, SF1777, SF1670, SF1674 and SF1770 was tested using the MTT assay in 3 cell lines including human brain endothelial cells (HBEC), human prostate cancer cells (PC-3) and human non-small cell lung cancer cells (H1299). Cells were plated into 96-well plates at 10,000-20,000/100 ul/well in RPMI 1640 medium supplemented with 10% FBS and incubated overnight at 37° C. in an incubator containing an atmosphere of 5% CO₂. The following day, the medium was replaced and cells were starved by placing in 100 uL of serum-free medium for 3 hours. Serially diluted test compounds were added to the wells and incubated with the cells for 2 hours at 37° C. Compounds were tested in a range from 1 mM to 0.1 nM depending on solubility. MTT was added to the wells at a final concentration of 5 μg/ml and incubated with the cells for 3 more hours. At the end of the incubation, the medium was aspirated and the MTT stain in the cells was dissolved by the addition of 100 μl DMSO. Optical density of each well was then measured at 570 nm using a SpectroMax Plus spectrophotometric plate reader (Molecular Devices, Sunnyvale, Calif.). The IC50 determination from the data was made using Prism software (GraphPad Software, San Diego, Calif.). The table below shows the IC50 (uM) of the PTEN inhibitors tested in this manner:

TABLE 1
Viability IC50 of PTEN inhibitors:

| | | | | PTEN Inhibition | |
|---|---|---|---|---|---|
| | HBEC (uM) | H1299 (uM) | PC-3 (uM) | IC50 | |
| SF1670 | 5 | 10 | 44 | 2.0 | uM |
| SF1674 | 133 | 188 | 154 | 33 | nM |
| SF1770 | >1000 | 695 | >1000 | 2.2 | uM |
| SF1720 | 1 | <1 | 7 | 5.0 | uM |
| SF1773 | 2 | <1 | >1000 | 297 | nM |
| SF1777 | <1 | 17 | 13 | 292 | nM |

These results show that several PTEN inhibitors have a large therapeutic window with the concentration of compound needed to adversely affect cells being much greater than the concentration needed to inhibit 50% of PTEN's phosphatase activity.

### EXAMPLE 9

### In Vitro Aortic Ring Angiogenesis Assay

The ability of the PTEN inhibitors to stimulate the angiogenic process was tested in vitro using this mouse aortic ring matrigel angiogenesis model [Burbridge M F et al, Rat Aortic Ring: 3D model of Angiogenesis in Vitro. Page 185, in Angiogenesis Protocols, Edited by J. Clifford Murray, 2000]. Thoracic aortas were harvested from nude mice and cut into 1-1.5 mm rings under a stereo microscope. Aortic rings were rinsed in serum-free DMEM medium 3 times and laid atop a 100 μl pad of Matrigel in wells of a 96-well plate (one ring per well). 50 ul of serum-free medium only (as control), or medium containing 5 μM of PTEN inhibitors (test solutions), or medium containing 40 μM of LY294002 was then added to the wells on top of the rings. The rings, partially embedded in the matrigel and exposed to the solution on top were then allowed to incubate at 37° C. in an incubator for 5 days. The rings embedded in the matrigel were then viewed under an optical microscope at 40× magnification and the number of direct “stems” growing out from the rings and the number of “branches”, which are appendages coming off of the “stems” were counted. The range for the total numbers of the stems and branches shown in the table below were averages of duplicate treatments from two separate experiments and are normalized relative to the negative control (no test compound). LY29002 was used as an additional negative control, since it has been reported in the literature to have an anti-angiogenic effect. These results clearly show that PTEN inhibitors can stimulate the angiogenic processes.

TABLE 2

| | Stem (% of control) | Branch (% of control) |
|---|---|---|
| SF1740 | 219-228 | 227-260 |
| SF1773 | 100-367 | 94-330 |
| SF1779 | 92-125 | 112-138 |
| SF1674 | 164-201 | 164-198 |
| SF1670 | 107-196 | 126-166 |
| Control | 100 | 100 |
| LY294002* | 0-2 | 0-0.8 |

### EXAMPLE 10

### General Migration Assay

A Migration Assay was performed using Costar transwells (Corning Costar, Cambridge, Mass.) possessing 8 um pore size to test the migration capacity of glioma cells U87MG (PTEN null) and glioma cells with PTEN genetically reconstituted U87MG/PTEN (Su et al., Cancer Research 2003, Vol. 63, pps. 3585-3592), mouse embryo fibroblast MEF (naturally containing PTEN=PTEN wild type=wt), along with PTEN genetically knocked out of MEF (MEF PTEN null) in the presence and absence of PTEN inhibitors. Prior to adding cells, the bottom side of the upper cup of the transwell was coated with 10 ug/ml vitronectin (BD Biosciences, Bedford, Mass.) and incubated at 37° C. for 1 hour. Cells were serum pre-starved (using serum-free media) overnight. Adherent cells were trypsinized with Typsin-EDTA (Invitrogen, Carlsbad, Calif.), and then 2 million cells/cup were added into the vitronectin-coated upper cups of the transwell. Then a 600 ul portion of serum free media containing various doses of PTEN inhibitors was added into each lower chamber of the transwell. The whole transwell was then allowed to incubate at 37° C. in a 5% CO₂ atmosphere for 4 hours. The upper cup of the transwell (both sides of the the membrane) was then stained with 1 mg/ml crystal violet, 50 mM boric acid, 15 mM borex (All reagents from Aldrich, Milwaukee, Wis.) at room temperature overnight. The cup was rinsed with water and the upper side of the cup was wiped with a cotton swab and the number of stained cells on the bottom side of the transwell cup were then counted under the microscope. Several experiments were run in duplicate. Each stained transwell cup bottom was examined by five random views under the microscope so that each migration datapoint comes from ten values to arrive at an average migration number along with a standard deviation (Stdev). These numbers were then compared to the “0” concentration of inhibitor number to arrive at statistical significance which is presented as a p value less than or equal to 0.05.

TABLE 3

| compound | genotype | function | 0 uM | 10 uM | 30 uM | 60 uM | 120 uM | 250 uM |
|---|---|---|---|---|---|---|---|---|
| SF1670 | MEF PTEN wt | Average | 15.40 | 16.00 | 22.60 | 31.20 | 28.70 | 26.80 |
| | | Stdev | 1.26 | 1.89 | 2.46 | 3.22 | 3.59 | 2.78 |
| | | p | | 0.41 | <0.05 | <0.05 | <0.05 | <0.05 |
| | MEF PTEN | Average | 51.50 | 47.20 | 49.30 | 49.90 | 47.60 | 45.90 |
| | null | Stdev | 6.28 | 6.70 | 7.45 | 6.71 | 8.36 | 9.30 |
| | | p | | 0.16 | 0.48 | 0.59 | 0.25 | 0.13 |
| SF1740 | MEF PTEN wt | Average | 16.20 | 18.20 | 20.60 | 28.30 | 30.80 | 32.10 |
| | | Stdev | 3.12 | 2.49 | 2.63 | 3.47 | 3.22 | 3.25 |
| | | p | | 0.13 | <0.05 | <0.05 | <0.05 | <0.05 |
| | MEF PTEN | Average | 51.60 | 50.70 | 56.10 | 52.50 | 54.10 | 48.40 |
| | null | Stdev | 6.17 | 8.56 | 5.90 | 6.31 | 8.20 | 5.74 |
| | | p | | 0.79 | 0.11 | 0.75 | 0.45 | 0.25 |

TABLE 4

| Compound | genotype | function | 0 uM | 125 uM | 250 Um |
|---|---|---|---|---|---|
| SF1674 | MEF PTEN | Average | 10.50 | 13.50 | 13.90 |
| | wt | Stdev | 3.06 | 1.84 | 2.64 |
| | | P | | <0.05 | <0.05 |
| | MEF PTEN | Average | 42.80 | 40.50 | 43.70 |
| | null | Stdev | 4.49 | 6.75 | 6.29 |
| | | P | | 0.38 | 0.72 |
| SF1720 | MEF PTEN | Average | 10.50 | 18.20 | 11.90 |
| | wt | Stdev | 3.06 | 2.66 | 2.13 |
| | | P | | <0.05 | 0.25 |
| | MEF PTEN | Average | 42.80 | 36.50 | 37.30 |
| | null | Stdev | 4.49 | 4.74 | 6.04 |
| | | P | | <0.05 | <0.05 |
| SF1770 | MEF PTEN | Average | 10.50 | 22.90 | 22.40 |
| | wt | Stdev | 3.06 | 2.85 | 2.88 |
| | | P | | <0.05 | <0.05 |
| | MEF PTEN | Average | 42.80 | 42.30 | 41.50 |
| | null | Stdev | 4.49 | 8.12 | 5.82 |
| | | P | | 0.87 | 0.58 |
| SF1773 | MEF PTEN | Average | 10.50 | 28.50 | 17.50 |
| | wt | Stdev | 3.06 | 2.17 | 2.59 |
| | | P | | <0.05 | <0.05 |
| | MEF PTEN | Average | 42.80 | 36.80 | 38.10 |
| | null | Stdev | 4.49 | 7.02 | 3.63 |
| | | P | | <0.05 | <0.05 |
| SF1779 | MEF PTEN | Average | 10.50 | 20.60 | 18.00 |
| | wt | Stdev | 3.06 | 1.96 | 2.67 |
| | | P | | <0.05 | <0.05 |
| | MEF PTEN | Average | 42.80 | 47.30 | 43.50 |
| | null | Stdev | 4.49 | 4.79 | 6.92 |
| | | P | | <0.05 | 0.79 |

The PTEN null cells by having PTEN genetically surpressed, mutated, or not present yield highly migrating cells. The PTEN containing cells are much lower in their migrating propensity due to the influence that active PTEN has in controlling cellular vitronectin mediated-migration (note “0” concentration of inhibitor is the basel migration data). In all cases the PTEN inhibitors increase the degree of migration of the PTEN-possessing cells consistent with the intracellular inhibition of PTEN in these cells making them behave more like the cells with genetically inactive PTEN.

Similar results below using U87MG glioma cells show that there is an optimal PTEN inhibitor concentration for inducing the migration phenotype and in both cases (SF1670 and SF1740) maximal induction of migration occurs at a concentration of 30 nM. Higher concentrations of inhibitors are not statistically significant relative to controls which further supports a maximum biological effectiveness of around 30 nM. It should be noted that the inhibitors had no significant effect on the PTEN null cells as expected.

TABLE 5

Concentration of PTEN inhibitor candidates (nM)

| Compound | Genotype | Function | 0 | 10 | 30 | 60 | 120 | 250 |
|---|---|---|---|---|---|---|---|---|
| SF1670 | U87MG | Average | 23.90 | 33.40 | 53.70 | 35.80 | 24.10 | 27.30 |
| | PTEN wt | Stdev | 6.23 | 4.20 | 12.04 | 10.60 | 2.51 | 6.15 |
| | | p | | <0.05 | <0.05 | <0.05 | 0.93 | 0.23 |
| | U87MG | Average | 58.80 | 60.90 | 57.20 | 57.20 | | |
| | PTEN null | Stdev | 9.34 | 9.33 | 10.56 | 7.36 | | |
| | | p | | 0.62 | 0.72 | 0.68 | | |
| SF1740 | U87MG | Average | 17.60 | 29.40 | 34.60 | 22.40 | 18.20 | 19.10 |
| | PTEN wt | Stdev | 4.62 | 5.17 | 4.84 | 4.20 | 3.46 | 3.07 |
| | | p | | <0.05 | <0.05 | <0.05 | 0.75 | 0.40 |
| | U87MG | Average | 56.80 | 58.10 | 54.80 | 56.70 | 56.10 | 54.10 |
| | PTEN null | Stdev | 5.57 | 6.28 | 5.29 | 6.34 | 6.57 | 5.84 |
| | | p | | 0.63 | 0.42 | 0.97 | 0.80 | 0.30 |

### EXAMPLE 11

### General Wound Healing Assay

The wound-healing assay was employed to determine the rate at which a leading edge of cells migrate outward. Multiple 6 cm tissue culture dishes were used to test the healing speed with or without PTEN inhibitors added into the medium. Six horizontal lines and one crossing vertical line were drawn on the bottom of each of the 6 cm tissue culture dishes with a fine Sharpie marker. Two million HBEC (Human Brain Endothelial Cells) were plated into each of the marked dishes. The plated cells were incubated at 37° C. in an atmosphere of 5% CO₂ for 24 hours. The “wound” was generated using a flat-edge of a cell scraper by aligning one edge of the scraper on the marked vertical line and moving the scraper across all six horizontal lines creating a gash in the confluent cell layer that we call a “wound” approximately 1 cm wide. The cells were washed in the dishes with PBS to remove debris and then the media was replaced with media containing the PTEN inhibitors dissolved therein. Then, under a microscope with an optic reticule, the distance the cells grown out from the initial wound edge was measured along the six crossing points of the vertical and horizontal tracking lines. Thus each dish yielded 6 data points. After 2, 4, 6 and 24 hours of incubation at 37° C. in an atmosphere of 5% CO₂, the distance the cells have grown out at the same location along the tracking lines was measured under the microscope and presented are in millimeters.

TABLE 6
PTEN inhibitors effect on in vitro wound healing model

| | | 2 hrs | 4 hrs | 6 hrs |
|---|---|---|---|---|
| No treatment control | | 0.003333 | 0.013333 | 0.031667 |
| SF1720 | 0.111 uM | 0.043333333 | 0.056666667 | 0.068333333 |
| | 0.333 uM | 0.05 | 0.085 | 0.133333333 |
| SF1740 | 0.111 uM | 0.046666667 | 0.071666667 | 0.071666667 |
| | 0.333 uM | 0.036666667 | 0.071666667 | 0.088333333 |

Each data point is the average of six to nine different measurements of the distance the wound edge had moved forward, in mm. All compound treatment samples in the table above have statistical difference (p<0.05) from the “no treatment control” for each time point. These results clearly show that relative to the control, PTEN inhibitors (SF1720 and SF1740) induce cells (HBEC) to migrate faster.

The above experiment was repeated at lower concentrations and examining other PTEN inhibitors (SF1670,SF1770 and SF1773). These results show that PTEN inhibitors SF1670, SF1770 and SF1773 induce HBEC cells to migrate faster across an artificially created gap (“wound”) in vitro.

TABLE 7

| | | | 2 hrs | 4 hrs | 6 hrs | 24 hrs |
|---|---|---|---|---|---|---|
| No treatment control | | | 0.005 | 0.008333 | 0.031667 | 0.108333 |
| 1670 | 10 | nM | 0.0233333 | 0.0616667 | 0.0683333 | 0.1516667* |
| | 50 | nM | 0.0583333 | 0.0633333 | 0.0733333 | 0.1966667 |
| | 250 | nM | 0.04166667 | 0.06333333 | 0.08333333 | 0.15* |
| 1720 | 10 | nM | 0.03* | 0.0466667 | 0.08* | 0.155* |
| | 50 | nM | 0.0366667 | 0.0466664 | 0.0566667* | 0.158333* |
| | 250 | nM | 0.025 | 0.03666667 | 0.05166667* | 0.11166667* |
| 1740 | 10 | nM | 0.0366667 | 0.065 | 0.085 | 0.1616667* |
| | 50 | nM | 0.05 | 0.0616667 | 0.0716667 | 0.1233333* |
| | 250 | nM | 0.043333333 | 0.06833333 | 0.098333333 | 0.2 |
| 1770 | 10 | nM | 0.0266667* | 0.045* | 0.065* | 0.1483333* |
| | 50 | nM | 0.05 | 0.06 | 0.075 | 0.2233333* |
| | 250 | nM | 0.04 | 0.05166667 | 0.06666667 | 0.20166667* |
| | 1000 | nM | 0.04 | 0.061667 | 0.076667 | 0.241667 |
| 1773 | 10 | nM | 0.0366667 | 0.0566667 | 0.0716667 | 0.2133333 |
| | 50 | nM | 0.0366667 | 0.0566667 | 0.0766667 | 0.2283333 |
| | 250 | nM | 0.02833333 | 0.05333333* | 0.075* | 0.22166667 |
| | 1000 | nM | 0.023333* | 0.03333* | 0.07 | 0.181667* |

*not statistically different from the “no treatment control” (p > 0.05)

### EXAMPLE 12

### Immobilized PTEN

In the PTEN inhibition assay, to exclude the possibility that PTEN inhibitor candidates interact with the unilameller PIP3 lipid vesicle (PLV) to block PTEN and artificially mimic PTEN inhibition, PTEN was bound to a solid support and treated with inhibitor, the inhibitor washed away and then the capability of the bound PTEN to dephosphorylate PLV was determined. In this approach, 100 ul of gel slurry (Gluthathione Sepharose 4B gel (Amersham, Piscataway, N.J.) was incubated and shaken with 200 ug of GST-PTEN fusion protein at room temperature for 3 hours. The gel was centrifuged, washed 3 times and centrifuged again. The gel was re-suspended in 100 mM Tris, pH 8.0, 10% glycerol, and stored at 4° C. The gel was then incubated with PTEN inhibitor (SF1720) at room temperature for 1 hour, centrifuged, washed 3 times, re-suspended, and then substrate (PLV) was added and the mix was incubated at 37° C. for 20 minutes. Then a 100 ul portion of malachite green buffer (Upstate, Charlottesville, Va.) was added to develop the color in the dark at room temperature. A SpectraMax Plus spectrophotometric plate reader (Molecular Devices, Sunnydale, Calif.) was used to measure the optical density at 650 nanometers due to the reaction of malachite green with liberated inorganic phosphate. Optical density measurements were converted to nanomoles of phosphate detected using a phosphate-malachite green standard calibration curve. The results are shown below (numbers are nanomoles of inorganic phosphate detected):

TABLE 8

| | | | | PTEN gel/ | PTEN gel/ |
|---|---|---|---|---|---|
| | | oriPTEN + | oriPTEN + | DMSO | 1720 |
| | PLV alone | DMSO | 1720 | bound | bound |
| | 0.0946 | 0.9253 | 0.0995 | 0.4471 | 0.1191 |
| | 0.086 | 0.9302 | 0.106 | 0.4324 | 0.1093 |
| Average | 0.0905 | 0.9277 | 0.1028 | 0.4398 | 0.1142 |
| Stdev | 0.0057 | 0.0034 | 0.0046 | 0.0103 | 0.0069 |

These results clearly show that the amount of phosphate liberated by immobilized PTEN is much greater than immobilized PTEN which has been pretreated with SF1720 for an hour (and then excess inhibitor washed away). The data in the first column is the amount of phosphate generated by the substrate alone (i.e. no PTEN added) and is essentially the background level of phosphate. The second column is the solution reaction of PTEN with PLV substrate with DMSO added (amount normally used to introduce the PTEN inhibitors) which shows the usual robust generation of inorganic phosphate from the dephosphorylation of PIP3 in the PLV substrate. The third column shows the inhibition of such PTEN dephosphorylation by the addition of SF1720 in DMSO down to essentially background amounts of phosphate. The fourth column shows the amount of phosphate liberated from PLV by non-inhibited PTEN bound to the Sepharose gel (immobilized). The fifth column shows the amount of phosphate liberated from PLV substrate by PTEN bound to the Sepharose gel wherein this PTEN had been previously exposed to the inhibitor SF1720 and washed extensively to remove any non-bound SF1720 inhibitor. The small quantity of phosphate found in the fifth column strongly suggest that the SF1720 inhibitor binds to PTEN and stays with the immobilized PTEN in the presence of washes and that such SF1720-PTEN interaction does not allow PTEN to significantly dephosphorylate the PLV substrate (i.e phosphate liberated in this part of the experiment was very close to background phosphate). This experiment demonstrates that the PTEN inhibitor may remain with the PTEN protein and the inhibition of PTEN is not due to interference with the substrate availability but is due to small molecule inhibitor interactions with the PTEN protein.

### EXAMPLE 13

### Kinetic Reaction of PTEN in Presence of Inhibitors

PTEN was mixed with substrate (PLV) in 3 groups differing according to the timing of adding 100 uM PTEN inhibitor, e.g. 1) no inhibitor added; 2) inhibitor added immediately and 3) inhibitor added after 10 minutes progression of PTEN reaction with substrate. Then after 0, 1, 5, 15, 20, 30, 60 minutes, aliquots of PTEN reaction mixture were added into the stop solution comprised of a 5× volume of 200 mM N-ethylmaleimide which reacts covalently with the catalytic sulphydryl group of PTEN and shuts off any additional dephosphorylation. Samples then were quantitated by exposure to malachite green and then measuring absorbance at OD650 nm to determine the amount of phosphate liberated. Optical density measurements were converted to nanomoles of phosphate detected using a phosphate-malachite green standard calibration curve. The results are shown below (experiment was performed in duplicate).

TABLE 9
Reaction Time (minutes)

| | 0 | 5 | 15 | 30 | 60 |
|---|---|---|---|---|---|
| No SF1589 treated | 0.400 | 0.749 | 1.219 | 1.180 | 1.262 |
| | 0.398 | 0..736 | 1.203 | 1.208 | 1.224 |
| 250 mM SF1589, added | 0.506 | 0.570 | 0.650 | 0.619 | 0.669 |
| immediatedly | 0.501 | 0.615 | 0.659 | 0.633 | 0.627 |
| 250 mM SF1589, added 10 min | 0.397 | 0..775 | 0.940 | 0.934 | 0.907 |
| after PTEN reaction | 0.395 | 0..754 | 0.906 | 0.914 | 0.934 |

The results show that PTEN liberates phosphate at a high rate for about 15 minutes and then stops (control-top entry). In the continuous presence of the PTEN inhibitor SF1589 from the start of the reaction (middle entry) the amount of phosphate generated is greatly diminished at all time points relative to the control experiment. The addition of the PTEN inhibitor after 10 minutes (bottom entry) into the reaction clearly shows that the PTEN is actively producing inorganic phosphate until the inhibitor is added at which time it levels off a much lower level than the negative control sample indicating that as soon as inhibitor was added it did indeed inhibit further dephosphorylation of the PLV substrates.

### EXAMPLE 14

### Vanadate-Based PTEN Inhibitors

Commercially available vanadates (EMD Biosciences, Inc.) were screened in the PTEN assay according to the method of Example 3 at 250 uM concentrations and were found to inhibit phosphorylation, as shown in Table 10. PTEN hydrolyzes phosphate at the 3 position on the inositol ring of PtdIns(3,4,5)P3, and Ins(1,3,4,5)P4. The release of phosphate from the natural substrate was measured in a colorimetric assay by using the Malachite Green Reagent (Upstate) in accordance with the instructions of the manufacturer. The absorbance at 650 nm was recorded in an ELISA plate reader. A standard curve was performed in each assay, and the amount of free phosphate was calculated from the standard curve line-fit data.

TABLE 10

| | | % Inh. |
|---|---|---|
| | | @ 250 |
| | | uM in |
| | | PTEN |
| Compound Name | SF Number | Assay |
| Potassium Bisperoxo(bipyridine)oxovanadate (V) | SF1668-101 | 94.7 |
| Dipotassium Bisperoxo(5-hydroxypyridine-2- | SF1674-101 | 98-97.2 |
| carboxyl)oxovanadate (V) | | |
| Dipotassium Bisperoxo(picolinato)oxovanadate | SF1675-101 | 98.1 |
| (V) | | |
| Monoperoxo(picolinato)oxovanadate(V) | SF1677-000 | 96.9 |
| Potassium Bisperoxo(1,10- | SF1678-101 | 100.4 |
| phenanthroline)oxovanadate (V) | | |
| bis(N,N- | SF1682-000 | 67.9 |
| Dimethylhydroxamido)hydroxooxovanadate | | |

The vanadate compounds were screened in a PTP1B assay using both synthetic GluTyr substrate (a 10-mer of (Glu₄Tyr[P])₂ synthesized in house) as well as p-nitrophenylphosphate (pNPP). Each experiment was performed in triplicate.

Using the assay conditions reported herein, the bpVs did not display selective inhibition for PTEN over PTP1B. In addition, the inhibition observed in the PTEN assay was approximately an order of magnitude less potent then reported in the literature [Schmid, A. C.; Byrne, R. D.; Vilar, R.; Woscholski, R., Bisperoxovanadium compounds are potent PTEN inhibitors. FEBS Lett 2004, 566, (1-3), 35-8] (see chart below).

TABLE 11

| | | | | Semafore | | |
|---|---|---|---|---|---|---|
| | | ImperialCollege* | | Pharmaceuticals** | | |
| Compounds | SF Number | PTEN | PTP1B | PTEN | PTP1B | PTP1B |
| bpV(bipy) | SF1668-101 | 18 nM ± 0.8 | 164 nM ± 22.6 | 276.3 nM ± 36.6 | 103 nM | 213.7 uM ± 27.1 |
| bpV(phen) | SF1678-101 | 38 nM ± 2.4 | 920 nm ± 45.2 | 356.6 nM ± 91.4 | 83.98 nM | 75.43 nM |
| bpV(HOpic) | SF1674-101 | 14 nM ± 2.3 | 25.3 uM ± 2.9 | 91.1 nM ± 6.4 | 45 nM | 79.5 nM ± 22.1 |
| bpV(pic) | SF1675-101 | 31 nM ± 1.7 | 61 uM ± 10.5 | 111.2 nM ± 6.4 | 82 nM | 145.8 nM |
| | SUBSTRATE | PtdIns(3, 4, 5)P₃ | pNPP | PtdIns(3, 4, 5)P₃ | (Glu₄Tyr[P])₂ | pNPP |
| | | | | Triplicate | triplicate | triplicate |

*Schmid, A. C.; Byrne, R. D.; Vilar, R.; Woscholski, R., Bisperoxovanadium compounds are potent PTEN inhibitors. FEBS Lett 2004, 566, (1-3), 35-8
**Garlich J. R. Small Molecule PTEN Inhibitors: Therapeutic Applications and Implications, in Signal Transduction Targets for Effictive Therapeutics, 2004, Boston, MA.

### EXAMPLE 15

### Evaluation of 4-hydroxynonenal as a PTEN Inhibitor

It has been reported that 4-hydroxynonenal (HNE), a ubiquitous by-product of fatty acid peroxidation heretofore shown to be carcinostatic inhibits PTEN activity in vitro at micromolar concentrations [Salsman, S. J. H., Kenneth; Floyd, Robert A. In 4-Hydroxynoneal inhibits PTEN phosphatase in vitro, 2003; Proceeding of the AACR, Vol. 44, 2^{nd} ed. July 2003]. Using the assay conditions reported herein, HNE did not display significant PTEN inhibition activity at 250 uM.

TABLE 12

| | | % Inhibition |
|---|---|---|
| Compound Name | CCTI Number | @ 250 uM in PTEN Assay |
| 4-hydroxy nonenal | CC1528-000 | −47 |

### EXAMPLE 16

### Evaluation of Alendronate

Alendronate (4-amino-1-hydroxybutylidene-1,1-bisphosphonate) is a potent bisphosphonate that inhibits osteoclastic bone resorption and has proven effective for the treatment of osteoporosis [Skorey, K.; Ly, H. D.; Kelly, J.; Hammond, M.; Ramachandran, C.; Huang, Z.; Gresser, M. J.; Wang, Q., How does alendronate inhibit protein-tyrosine phosphatases? J Biol Chem 1997, 272, (36), 22472-80.; Schmidt, A.; Rutledge, S. J.; Endo, N.; Opas, E. E.; Tanaka, H.; Wesolowski, G.; Leu, C. T.; Huang, Z.; Ramachandaran, C.; Rodan, S. B.; Rodan, G. A., Protein-tyrosine phosphatase activity regulates osteoclast formation and function: inhibition by alendronate. Proc Natl Acad Sci USA 1996, 93, (7), 3068-73]. It has also been reported as a potent inhibitor of the protein-tyrosine-phosphatase-meg1 (PTPmeg1) [Opas, E. E.; Rutledge, S. J.; Golub, E.; Stern, A.; Zimolo, Z.; Rodan, G. A.; Schmidt, A., Alendronate inhibition of protein-tyrosine-phosphatase-meg1. Biochem Pharmacol 1997, 54, (6), 721-7]. Using the assay conditions reported herein, aledronate was screened and found to have minimal activity at 250 uM. These results show that although alendronate is touted as a significant phosphatase inhibitor it does not reproducibly and significantly inhibit PTEN.

TABLE 13

| | CCTI | % Inhibition @ |
|---|---|---|
| Compound Name | Number | 250 uM in PTEN Assay |
| Aledronate, Sodium, Trihydrate | CC1669-100 | −0.9 to 20 |

### EXAMPLE 17

### PTEN Inhibitors Based on Triazoles

Commercially available compounds from a chemical procurement company (ChemNavigator®) were tested in the PTEN assay reported herein with the results shown below. These compounds comprise a core triazole ring directly linked to a furazan ring, and are of the following formula.

TABLE 14

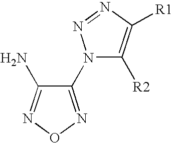

Substituted Triazole Series

| Compound | | | % Inhibition |
|---|---|---|---|
| Number | R1 Group | R2 Group | at 250 uM |
| SF1512-000 | CH₃ | C(Me)NNH₂ | 6.8 |
| SF1515-000 | CONHNCHPh(4Br) | CH₂NHPh | 38-56 |
| SF1523-000 | CONHNCHPh(3-OEt, 4-O | CH₂OPh | 53-85 |
| | CH₂CONH₂) | | |
| SF1623-001 | CONHNCHPh(3-NO2) | CH₂NHPh | −50 to 16 |
| SF1635-000 | CO₂Et | CH₂S-2-benzo | 21.2 |
| | | [d]thiazole | |
| SF1636-000 | CO₂Et | CH₂-indoline | 17.5 |
| SF1640-000 | CO₂Et | CH₂S(1H- | 2.1 |
| | | benzo[d] | |
| | | imidazol-2-yl) | |
| SF1644-000 | CO₂Et | CH₂NHPh | 12.4 |
| | | (4-OMe) | |
| SF1726-000 | CO₂Et | NH CH₂Ph | 25.4 |
| | | (3-Br, 4-Me) | |
| SF1728-000 | CONHNCHPh(3,4-diOMe) | Me | 34 |

### EXAMPLE 18

### Diamide Series—Furazan Core

The Diamide Series started out as a symmetrical molecule with a core ring system comprised of a furazan ring (SF 1518). Derivatives were synthesized to determine the inhibitory effect of the symmetrical R groups, the core ring system, and the symmetry of the molecules. The compounds in the Table below show the examples of R groups attached to the core furazan ring via an amide bond. The percent inhibition in the PTEN assay of Example 3 at 250 micromolar was obtained.

TABLE 15

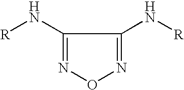

| | | | % | |
|---|---|---|---|---|
| | | Mol. | Inhibition @ | Ret. |
| SF Number | R | Wt | 250 uM | Time |
| SF1518-000 | COCH₂OPh(3-Me) | 396.4 | 46.6 | No Data+ |
| SF1607-000 | COCH₂OPh(2-OMe) | 428.4 | 11 | No Data+ |
| SFI608-000 | COCH₂OPh(4-Br) | 526.14 | 26-42 | No Data+ |
| SF1609-000 | COCH₂OPh(2,5-diMe) | 424.45 | 58-80 | No Data+ |
| SF1610-000 | COCH₂OPh | 480.56 | 28-39 | No Data+ |
| | (2-iPr,5-Me) | | | |
| SF1611-000 | COCH₂OPh(4-OMe) | 428.4 | 20-34 | 3.79 |
| SF1612-000 | COCH₂Oph | 368.34 | 82 | No Data+ |
| SF1614-000 | COCH₂OPh(2-Me) | 396.4 | 42-56 | No Data+ |
| SF1615-000 | COCH₂OPh(4-Me) | 396.4 | 38-46 | No Data+ |
| SF1616-000 | COCH₂OPh(2-iBu) | 480.56 | 13-41 | No Data+ |
| SF1617-000 | COCH₂OPh(2-iPr) | 452.4 | 3-81 | 4.94 |

+Purchased from Vitas-M Laboratory, LTD

1. General Procedure for the Synthesis of the Diamide Series

Excess thionyl chloride was added to two equivalents of substituted phenoxyacetic acid and was stirred for 12 h. The reaction was concentrated and the corresponding acid chloride was added to a solution of one equivalent aromatic diamine and two equivalent of diisopropylethyl amine in anhydrous methylene chloride and stirred for 12 h. The reaction mixture was washed with 1N HCl, 10% w/w NaHCO₃ and dried (MgSO₄), and concentrated in vacuo. The crude reaction was purified by flash chromatography (SiO₂, methylene chloride/methanol (98:2). The product identity and purity was confirmed by electrospray LC-MS using method A.

### EXAMPLE 19

### Diamide Series—Non Aromatic Cyclic Core-Based PTEN Inhibitors

A representative example using disubstituted cyclohexane as the core ring system was prepared and tested for PTEN inhibition as shown below:

TABLE 16

| | |
|---|---|
| | SF1647 |
| 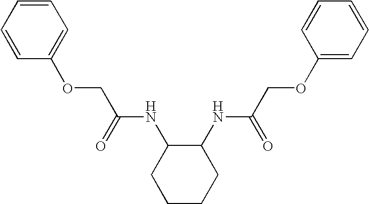 | |

| | Mol. | % Inhibition @ | |
|---|---|---|---|
| SF Number | Wt. | 250 uM | Retention Time |
| SF1647-000 | 382.45 | 2.2 | 3.61 |

Compound SF1647 was prepared by the following procedure: To 3.25 mmol 1,2 diaminocyclohexane (1 equiv.) in 5 mL methylene chloride was added 6.57 mmol phenoxyacetyl chloride (2 equiv.) (synthesized from the corresponding substituted phenol and chloroacetic acid as in Vogel, A. I.; Furniss, B. S.; Vogel, A. I., Vogel's Textbook of practical organic chemistry. 5th ed.; Longman Scientific & Technical: New York, 1989; p 968), 6.57 mmol diisopropylethyl amine (2 equiv.) and stirred over night. The reaction was concentrated in vacuo and purified via SiO₂ flash chromatography (methylene chloride/methanol, 98:2) to yield 0.04 g of desired product, LC/MS showed desired peak in >90% purity with Rt=3.61.

### EXAMPLE 20

### Diamide Series—Aromatic Phenyl Ring Core

A library replacing the core furazan ring with a phenyl ring was synthesized. The compounds in the following table show the examples of R groups attached to the core phenyl ring via an amide bond. The compounds were synthesized using the general procedure in Example 18. The substituted phenoxyacetic acid was synthesized from the corresponding substituted phenol and chloroacetic acid. The product purity and identity was confirmed by electrospray LC-MS using method A. In these examples the products were identified by either a [M+H]+ positive ion or a [M−H]− negative ion corresponding with the ultraviolet (UV) detected peak or the ELS (evaporative light scattering detector) peak.

TABLE 17

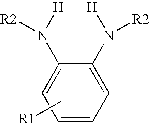

| | | | % | | |
|---|---|---|---|---|---|
| | | | Inhibition @ | Mol. | Ret. |
| SF Number | R1 | R2 | 250 uM | Wt. | Time |
| SF1632-000 | H | COCH₂OPh | 31.5 | 376.41 | 3.79 |
| SF1648-000 | H | COCH₂OPh | −15.5-4.9 | 460.64 | 5.04 |
| | | (2-iPr) | | | |
| SF1649-000 | H | COCH₂OPh | −13.2, −22.5 | 436.46 | 3.56 |
| | | (2-OMe) | | | |
| SF1695-000 | H | COCH₂OPh | 13-40 | 436.46 | 4.21 |
| | | (4-OMe) | | | |
| SF1701-000 | H | COCH₂OPh | 33-51 | 404.46 | 4.31 |
| | | (2-Me) | | | |
| SF1703-000 | H | COCH₂OPh | 32.8 | 436.46 | 3.86 |
| | | (3-OMe) | | | |
| SF1712-000 | H | COCH₂OPh | 15-69 | 436.46 | 3.75 |
| | | (4-OMe) | | | |
| SF1646-000 | 4-Me | COCH₂OPh | 1.8-35 | 390.43 | 4.02 |
| SF1696-000 | 4-CO₂H | COCH₂OPh | 29.2 | 420.41 | 5.61 |
| SF1697-000 | 3-Me | COCH₂OPh | 9.1 | 390.43 | 5.31 |
| SF1713-000 | 4-Me | COCH₂OPh | 18-66 | 450.46 | 3.90 |
| | | (4-OMe) | | | |
| SF1744-000 | 4CO₂H | COCH₂OPh | −18 | 480.47 | 3.45 |
| | | (4-OMe) | | | |

### EXAMPLE 21

### Diamide Series—Heteroaromatic Ring Core

A library replacing the core furazan ring with other aromatic rings was synthesized specifically incorporating pyridyl and pyrimidyl rings.

TABLE 18

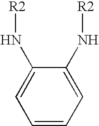

Heteroaromatic

| | Center | | % Inhibition @ |
|---|---|---|---|
| SF Number | Ring | R2 | 250 uM |
| SF1698-000 | Pyrimidine | COCH₂OPh | 36-38 |
| SF1747-000 | Pyridine*s | COCH₂OPh(4-OMe) | −33.5 |

*2,3-disubstituted pyridine

The compounds were synthesized using the general procedure in Example 18. The substituted phenoxyacetic acid was synthesized from the corresponding substituted phenol and chloroacetic acid [Vogel, A. I.; Furniss, B. S.; Vogel, A. I., Vogel's Textbook of practical organic chemistry. 5th ed.; Longman Scientific & Technical: New York, 1989; p 986]. The product identity and purity was confirmed by electrospray LC-MS using method A.

### EXAMPLE 22

### Monoamide Series—Furazan Ring Core

The effect of mono substitution on the core ring system was examined. Monosubstituted furazan analogs similar to those in Example 18 were synthesized. The synthesis was the same as in the previous example except for the use of equal stoichiometry of reactants. The product identity and purity was confirmed by electrospray LC-MS using method A.

TABLE 19

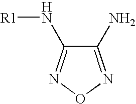

| | | | % Inhibition @ |
|---|---|---|---|
| SF Number | Core Ring | R1 | 250 uM |
| SF1748-000 | Furazan | COCH₂OPh(4-OMe) | −11.9 |

### EXAMPLE 23

### Monoamide Series—Phenyl Ring Core

The effect of mono substitution on the core ring system was examined. Free amino phenyl analogs were synthesized comparable to those synthesized in Example 22. The synthesis was the same as in the previous examples except for using equal stoichiometry of reactants to ensure that mainly one amine is acylated. The product identity and purity was confirmed by electrospray LC-MS using method A.

TABLE 20

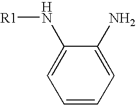

| | | | | % | |
|---|---|---|---|---|---|
| | | | | Inhibi- | |
| | | | Molec- | tion | HPLC |
| | Core | | ular | @ | Ret. |
| SF Number | Ring | R1 | Weight | 250 uM | Time |
| SF1700-000 | benzene | COCH₂OPh | 269.3 | −18.4 | 3.36 |
| | | (2-iPr) | | | |
| SF1704-000 | benzene | COCH₂OPh | 272.3 | 57-96 | 2.96 |
| | | (3-OMe) | | | |
| SF1706-000 | benzene | COCH₂OPh | 284.35 | 10.6 | 3.83 |
| | | (2-iPr) | | | |
| SF1710-000 | benzene | COCH₂OPh | 256.3 | 4.6 | 3.20 |
| | | (4-Me) | | | |
| SF1711-000 | benzene | COCH₂OPh | 272.3 | −13.6 | 2.86 |
| | | (4-OMe) | | | |
| SF1742-000 | benzene | COCH₂OPh | 286.33 | −21.9 | 3.04 |
| | (3-Me) | (4-OMe) | | | |
| SF1745-000 | benzene | COCH₂OPh | 316.31 | −25.4 | 2.55 |
| | (5CO₂H) | (4-OMe) | | | |

### EXAMPLE 24

### Monoamide Series—Phenyl Ring Core

The effect of mono substitution on the core ring system was examined. Free amino phenyl analogs were synthesized comparable to those synthesized in Example 21. The synthesis was the same as in Example 18 except for equal stoichiometry of reactants. The product identity and purity was confirmed by electrospray LC-MS using method A.

TABLE 21

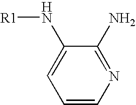

| | | | | % Inhi- | |
|---|---|---|---|---|---|
| | | | | bition | |
| | | | | @250 | Ret. |
| SF Number | Core Ring | R1 | Mol. Wt. | uM | Time |
| SF1746-000 | pyridine(2- | COCH₂OPh(4- | 273.29 | −3.5 | 1.46 |
| | NH2) | OMe) | | | |

### EXAMPLE 25

### 3-Carbonyl Imidazole Series

A library of aryl substituted 3-carbonyl imidazoles was synthesized examining the effect of substitution on the carbonyl group.

TABLE 22

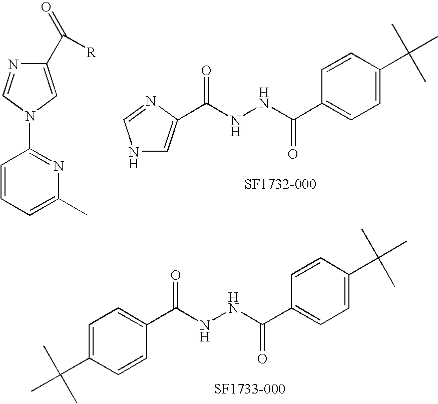

| | | | % | |
|---|---|---|---|---|
| | | | Inhibition | Ret. |
| SF Number | R Group | Mol. Wt. | at 250 uM | Time |
| SF1581-000 | NHNHSO₂(2-Naphthyl) | 407.45 | 41-96 | No |
| | | | | Data+ |
| SF1688-000 | NHNH₂ | 217.23 | 7.1 | 1.70 |
| SF1689-000 | NHNHCOPh(4-Me) | 335.37 | 6 | No |
| | | | | Data+ |
| SF1690-000 | NHNHCHPh(4-Br)# | 384.24 | 22.7 | No |
| | | | | Data+ |
| SF1691-000 | NHNHSO₂Ph(4-OMe) | 387.41 | 6.9 | No |
| | | | | Data+ |
| SF1692-000 | NHNCHPh(4-NO2)# | 350.34 | 39-45 | No |
| | | | | Data+ |
| SF1693-000 | NHNHSO₂Ph(3-CF₃) | 425.39 | 13.6 | 3.1 |
| SF1694-000 | NHNCHPh | 305.34 | 12.9 | No |
| | | | | Data+ |
| SF1699-000 | NHNHCO(2-naphthyl) | 371.40 | 41-43 | 3.1 |
| SF1702-000 | NHNHSO₂Ph(4-Me) | 371.42 | 43-46 | 3.1 |
| SF1707-000 | NHNHSO₂Ph(4-tBu) | 363.42 | 25.1 | 3.0 |
| SF1708-000 | NHNHCOPh(4-tBu) | 377.45 | 57-95 | 3.4 |
| SF1709-000 | NHNHCO(CH₂)₃)Ph | 377.45 | 28.5 | 3.7 |
| SF1714-000 | OH | 203.20 | 8 | 1.77 |
| SF1730-000 | NHNHCOPh(4-NO₂) | 366.34 | −9.7 | 2,7 |
| SF1731-000 | NHNHSO₂Ph(4-NO₂) | 402.39 | 8.3 | 3.1 |
| SF1732-000 | | 286.34 | | 2.6 |
| SF1739-000 | NHNCHPh(4-tBu)* | 361.45 | −14.3 | 4.0 |
| SF1775-000 | OMe | 217.23 | 18.3 | 2.4 |
| SF1733-000 | | 352.48 | 15.9 | 2.4 |

+commercially available (Bionet Research)

### EXAMPLE 26

### Preparation of SF1699-000

A solution of 10.0 mg 1-(6-methyl-2-pyridinyl)-1H-imidazole-4-carbohydrazide (Bionet Research, cat. no. 6P-707) in dichloromethane was treated with 2-naphthoyl chloride (1.2 eq) and diisopropylethyl amine (1.2 eq). The mixture was stirred overnight, diluted with dichloromethane and washed with 10% w/w sodium bicarbonate. The organics were dried over sodium sulfate, and the solvent removed to give 18.4 mg of a tan solid. The presence of the title compound was confirmed by electrospray LC-MS using method A; t_{R}=3.1 min. MS [M=C₂₁H₁₇N₅O₂] m/z 372 (MH⁺); 435 (MNa—CH₃CN⁺).

### EXAMPLE 27

### Preparation of SF1702-000

A solution of 10.0 mg 1-(6-methyl-2-pyridinyl)-1H-imidazole-4-carbohydrazide (Bionet Research, cat. no. 6P-707) in acetonitrile was treated with p-toluenesulfonyl chloride (1.2 eq) and diisopropylethyl amine (1.2 eq). The mixture was stirred overnight at 55° C., diluted with dichloromethane and washed with 10% w/w sodium bicarbonate. The organics were dried over sodium sulfate, and the solvent removed to give material that was subjected to silica gel chromatography using methanol-dichloromethane eluent, providing 2.5 mg of an oil. The presence of the title compound was confirmed by electrospray LC-MS using method A; t_{R}=3.1 min. MS [M=C₁₇H₁₇N₅O₃S] m/z 372 (MH⁺); 394 (MNa⁺); 435 (MNa—CH₃CN⁺).

### EXAMPLE 28

### Preparation of SF1707-000

SF1707-000 was prepared by a 2-step process. Step 1 (4-phenylbutanoyl chloride): A solution of 200 mg 4-phenylbutyric acid in dichloromethane was treated with oxalyl chloride (3.0 eq) and was stirred overnight. The solvent and excess oxalyl chloride was removed to give 227 mg of 4-phenylbutanoyl chloride as a clear oil. The presence of the title compound was confirmed by converting it to the methyl ester by dissolving a sample in methanol, and analyzing this by electrospray LC-MS using method A; t_{R}=3.7 min. The ester is UV-active-only (no ELS signal). This contrasts with the starting material which has an earlier retention time and is ELS-active.

Step 2: A solution of 15.0 mg 1-(6-methyl-2-pyridinyl)-1H-imidazole-4-carbohydrazide (Bionet Research, cat. no. 6P-707) in dichloromethane was treated with 4-phenylbutanoyl chloride (prepared above, 1.2 eq) and diisopropylethyl amine (1.2 eq). The mixture was stirred overnight, diluted with dichloromethane and washed with 10% w/w sodium bicarbonate. The organics were dried over sodium sulfate, and the solvent removed to give material that was subjected to silica gel chromatography using methanol-dichloromethane eluent, providing 8.5 mg of an oil. The presence of the title compound was confirmed by electrospray LC-MS using method A; t_{R}=3.0 min. MS [M=C₂₀H₂₁N₅O₂] m/z 364 (MH⁺); 386 (MNa⁺); 427 (MNa—CH₃CN⁺).

### EXAMPLE 29

### Preparation of SF1708-000

A solution of 12.0 mg 1-(6-methyl-2pyridinyl)-1H-imidazole-4-carbohydrazide (Bionet Research, cat. no. 6P-707) in dichloromethane was treated with 4-tert-butylbenzoyl chloride (1.2 eq) and diisopropylethyl amine (1.2 eq). The mixture was stirred overnight, diluted with dichloromethane and washed with 10% w/w sodium bicarbonate. The organics were dried over sodium sulfate, and the solvent removed to give material that was subjected to silica gel chromatography using methanol-dichloromethane eluent, providing 4.3 mg of a yellow solid. The presence of the title compound was confirmed by electrospray LC-MS using method A; t_{R}=3.4 min. MS [M=C₂₁H₂₃N₅O₂] m/z 378 (MH⁺); 400 (MNa⁺); 441 (MNa—CH₃CN⁺).

### EXAMPLE 30

### Preparation of SF1709-000

A solution of 12.0 mg 1-(6-methyl-2-pyridinyl)-1H-imidazole-4-carbohydrazide (Bionet Research, cat. no. 6P-707) in acetonitrile was treated with 4-tert-butylbenzenesulfonyl chloride (1.2 eq) and diisopropylethyl amine (1.2 eq). The mixture was stirred overnight at 50° C., diluted with dichloromethane and washed with 10% w/w sodium bicarbonate. The organics were dried over sodium sulfate, and the solvent removed to give material that was subjected to silica gel chromatography using methanol-dichloromethane eluent, providing 4.4 mg of an oil. The presence of the title compound was confirmed by electrospray LC-MS using method A; t_{R}=3.7 min. MS [M=C₂₀H₂₃N₅O ₃S] m/z 414 (MH⁺); 477 (MNa—CH₃CN⁺).

### EXAMPLE 31

### Preparation of SF1730-000

A solution of 12.0 mg 1-(6-methyl-2-pyridinyl)-1H-imidazole-4-carbohydrazide (Bionet Research, cat. no. 6P-707) in dichloromethane was treated with 4-nitrobenzoyl chloride (1.2 eq) and diisopropylethyl amine (1.2 eq). The mixture was stirred overnight, diluted with dichloromethane and washed with 10% w/w sodium bicarbonate. The organics were dried over sodium sulfate, and the solvent removed to give material that was subjected to silica gel chromatography using methanol-dichloromethane eluent, providing 1.1 mg of desired product. The presence of the title compound was confirmed by electrospray LC-MS using method A; t_{R}=2.7 min. MS [M=C₁₇H₁₄N₆O₄] m/z 367 (MH⁺); 408 (M−CH₃CN⁺); 430 (MNa—CH₃CN⁺).

### EXAMPLE 32

### Preparation of SF1731-000

A solution of 12.0 mg 1-(6-methyl-2-pyridinyl)-1H-imidazole-4-carbohydrazide (Bionet Research, cat. no. 6P-707) in acetonitrile was treated with 4-nitrobenzenesulfonyl chloride (1.2 eq) and diisopropylethyl amine (1.2 eq). The mixture was stirred overnight at 50° C., diluted with dichloromethane and washed with 10% w/w sodium bicarbonate. The organics were dried over sodium sulfate, and the solvent removed to give material that was subjected to silica gel chromatography using methanol-dichloromethane eluent, providing 4.8 mg of a yellow oil. The presence of the title compound was confirmed by electrospray LC-MS using method A; t_{R}=3.1 min. MS [M=C₁₆H₁₄N₆O₅S] m/z 403 (MH⁺); 444 (M−CH₃CN⁺).

### EXAMPLE 33

### Preparation of SF1732-000: Prepared by a 3-Step Process

SF1732-000 was prepared by a 3-step process. Step 1 (4-tert-butylbenzohydrazide): 4-tert-butylbenzoyl chloride (300 mg) was added to a solution of 766 μL (10 eq) hydrazine monohydrate in dichloromethane and the mixture was stirred 3 hr and then washed with 10% w/w sodium bicarbonate. The organics were dried over sodium sulfate, and the solvent removed to give 257 mg of 4-tert-butylbenzohydrazide as a white solid. The presence of the compound was confirmed by electrospray LC-MS using method A; t_{R}=3.1 min. MS [M=C₁₁H₁₆N₂O] m/z 403 (MH⁺); 444 (MH—CH₃CN⁺).

Step 2 (4-imidazole carbonyl chloride): A solution of 100 mg 4-imidazole carboxylic acid in acetonitrile was treated with thionyl chloride (4.0 eq) and was stirred 2 hr at 75° C. The solvent and excess thionyl chloride was removed to give 4-imidazole carbonyl chloride as a tan solid, which was used directly in the next step.

Step 3: Amide Formation: The 4-imidazole carbonyl chloride from step 2 was dissolved in acetonitrile treated with a solution of the 4-tert-butylbenzohydrazide (1.5 eq, from step 1) in dichloromethane. Triethylamine (1.2 eq) was added and the mixture was stirred overnight, diluted with dichloromethane and washed with 10% w/w sodium bicarbonate and saturated sodium chloride. The organics were dried over sodium sulfate, and the solvent removed to give material that was subjected to silica gel chromatography using methanol-dichloromethane eluent, providing 7.7 mg of a yellow solid. The presence of the title compound was confirmed by electrospray LC-MS using method A; t_{R}=2.6 min. MS [M=C₁₅H₁₈N₄O₂] m/z 287 (MH⁺); 450 (MNa—CH₃CN⁺).

### EXAMPLE 34

### Preparation of SF1733-000

In the preparation of SF1732-000, the chromatography step also provided 9.2 mg of the SF1733-000 as a white solid. This was produced in step 1 of the synthesis, and carried forward into step 3. The presence of the compound was confirmed by electrospray LC-MS using method A; t_{R}32 4.1 min. MS [M=C₂₂H₂₈N₂O₂] m/z 353 (MH⁺); 416 (MNa—CH₃CN⁺); 351 (M−H⁻).

### EXAMPLE 35

### Preparation of SF1739-000

A solution of 15.0 mg 1-(6-methyl-2-pyridinyl)-1H-imidazole-4-carbohydrazide (Bionet Research, cat. no. 6P-707) in acetonitrile was treated with 4-tert-butylbenzaldehye (3.0 eq) and a catalytic amount of p-toluenesulfonic acid. The mixture was stirred overnight, diluted with dichloromethane and washed with 10% w/w sodium bicarbonate. The organics were dried over sodium sulfate, and the solvent removed to give material that was subjected to silica gel chromatography using methanol-dichloromethane eluent, providing 9.0 mg of a white solid. The presence of the title compound was confirmed by electrospray LC-MS using method A; t_{R}=4.0 min. MS [M=C₂₁H₂₃N₅O] m/z 362 (MH⁺); 425 (MNa—CH₃CN⁺). The stereochemistry around the double bond was not determined.

### EXAMPLE 36

### Preparation of SF1775-000

This compound was prepared by a method based on that reported by Kiyomori, Marcoux, Buchwald, Tetrahedron Lett., 1999, p. 2647. Briefly, a reaction vessel was charged with methyl 4-imidazole carboxylate (1.5 eq); 1,10-phenanthroline (1.0 eq); trans, trans-dibenzylidene acetone (0.10 eq); and cesium carbonate (1.1 eq). Xylenes were added, followed by 2-bromo-6-methyl-pyridine (1.0 eq) and copper(II) trifluoromethane sulfonate (0.10 eq). The mixture was heated at 90° C. overnight, diluted in dichloromethane, and washed with saturated ammonium chloride and saturated sodium chloride. The organics were dried over sodium sulfate and the solvent removed to give a material that was purified by reverse-phase HPLC, yielding 4.4 mg of an off-white solid. The presence of the title compound was confirmed by electrospray LC-MS using method A; t_{R}=2.4 min. MS [M=C₁₁H₁₁N₃O₂] m/z 218 (MH⁺); 259 (MH—CH₃CN⁺); 281 (MNa—CH₃CN⁺).

### EXAMPLE 37

### Polyamide Series of PTEN Inhibitors

From broad screening of commercially available libraries several compounds were discovered with PTEN inhibitory activity as shown below:

TABLE 23

| Structure | SF Number | % Inhibition @250 uM |
|---|---|---|
| 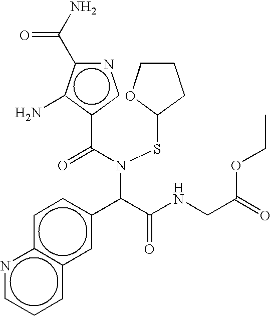 | SF1566-000 | 70.6 |
| 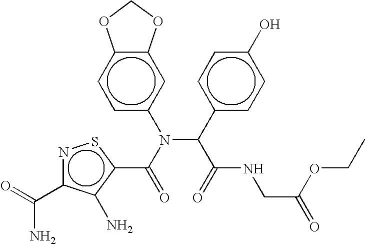 | SF1552-000 | 52.4 |
| 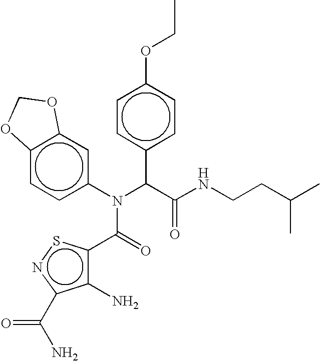 | SF1560-000 | 37.1 |
| 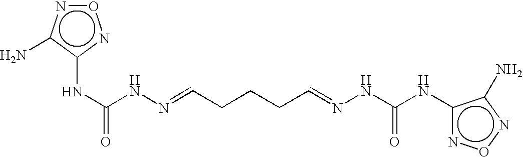 | SF1567-000 | 60.2 |
| 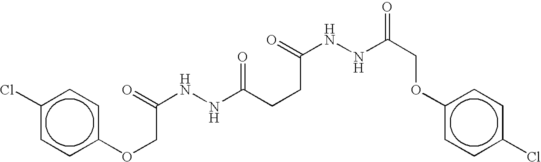 | SF1516-000 | 35.3 |

### EXAMPLE 38

### Commercially Available Known PTP Inhibitors Evaluated for PTEN Inhibition

Several known PTP inhibitors (obtained from EMD Biosciences, Inc) were tested for PTEN inhibition activity and the results are shown below:

TABLE 24

| Sample | | % Inhibition |
|---|---|---|
| Number | Compound Name | at 250 uM |
| SF1671-000 | 5-Benzyl-3-furylmethyl (1R,S)-cis,trans-chrysanthemate | 9.6 |
| SF1672-100 | Suramin, Sodium Salt; 8,8′-[carbonylbis[imino-3,1-phenylene carbonyl imino(4- | −9.9 |
| | methyl-3,1-phenylene)carbonylimino]]bis-, hexa sodium salt | |
| SF1673-000 | 4-Methoxyphenacyl Bromide | 2 |
| SF1676-000 | 1,4-Dimethylendothall; 1,4-Dimethyl-7-oxabicyclo[2.2.1]heptane-2,3-dicarboxylic | 3.3 |
| | Acid | |
| SF1679-000 | Cantharidic Acid; 2,3-dimethyl-7-oxa-bicyclo[2.2.1]heptane-2,3-di carboxylic acid | 2.7 |
| SF1680-000 | Sodium Stibogluconate; Antimony Sodium Gluconate | 1.5 |
| SF1681-000 | 3,4-Dephostatin, Ethyl- | 82.4 |
| SF1683-000 | Fenvalerate; a-Cyano-3-phenoxybenzyl-a-(4-chlorophenyl)isovalerate | −25.5 |
| SF1684-100 | α-Naphthyl Acid Phosphate, Monosodium Salt | 11.2 |
| SF1685-100 | β-Glycerophosphate, Disodium Salt, Pentahydrate | 10.9 |
| SF1686-000 | Endothall; 7-Oxabicyclo[2.2.1]heptane-2,3-dicarboxylic Acid | −1.2 |
| SF1687-000 | Cypermethrin; (R,S)-α-Cyano-3-phenoxybenzyl-3-(2,2-dichlorovinyl)-2,2-dimethyl | −5.9 |
| | cyclopropanecarboxylate; (1R)—(R)-cyano(3-phenoxy phenyl) methyl 3-(2,2- | |
| | dichloro vinyl)-2,2-dimethylcyclopropane carboxylate | |
| SF1667-000 | Deltamethrin; (S)-a-Cyano-3-phenoxybenzyl(1R)-cis-3-(2,2-dibromovinyl)-2,2- | 0.3 |
| | dimethyl cyclopropanecarboxylate | |

### EXAMPLE 39

### Phenanthroline Based PTEN Inhibitors

Commercially available 1,10-phenanthroline-5,6-dione (Aldrich) [SF1720 (R═H)] was evaluated in the PTEN assay and in the PTP1B assay and their respective IC50's determined. SF1720 showed more than 50 fold selective inhibition for PTEN over PTP1B.

TABLE 25

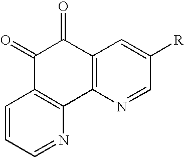

| SF Number | Substitution (R) | PTEN IC50 (uM) | PTPIB IC50 (uM) |
|---|---|---|---|
| SF1720-000 | H | 5 | ˜250 mM |

### EXAMPLE 40

### Phenanthrene Dione Based PTEN Inhibitors

Various phenanthrene-9,10-diones of the generic structure shown below were obtained or synthesized and tested in the PTEN inhibition assay described in Example 3.

2. General Procedure for the Synthesis of 3-Substituted Phenanthrene Diones

Hydrogenation of 3-nitrophenanthrene-5,6-dione using 10% Pd/Carbon in methanol over hydrogen gas produced the corresponding 3 aminophenanthrene-5,6-dione. This product was filtered using celite, concentrated in vacuo and used without further purification. Equal stoichiometry of amine, diisopropylethyl amine, and acid chloride in methylene chloride was stirred overnight. The reaction was washed with 10% HCl, 10% w/w sodium bicarbonate dried (MgSO₄) and concentrated in vacuo. The crude material was subjected to silica gel chromatography using methanol-dichloromethane eluent, or purified by preparative HPLC. The pure product was confirmed by electrospray LC-MS using method A. The substituted phenoxy acetyl chlorides used to make SF1740 and the like were synthesized from the corresponding substituted phenol (Aldrich) and chloroacetic acid by methods based on those reported in the literature (Vogel, A. I.; Furniss, B. S.; Vogel, A. I., Vogel's Textbook of practical organic chemistry. 5th ed.; Longman Scientific & Technical: New York, 1989; p xxviii, 1514).

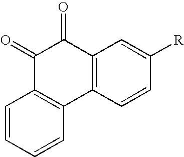

Several phenanthrene-9,10-diones were tested in the PTEN and PTP1B assay and the results are shown below

TABLE 26

| | | Mol. | % Inhibition | Ret. |
|---|---|---|---|---|
| SF Number | Substitution | Wt. | @ 250 uM | Time |
| SF1670-000 | NHCOC(CH₃)₃ | 307.3 | 100.3 | 3.7 |
| SF1721-000 | H | 208.21 | 99.7 | + |
| SF1722-000 | NO₂ | 253.21 | 99.3 | 3.47 |
| SF1740-000 | NHCOCH₂OPh | 357.37 | 98.4 | 3.9 |
| SF1751-000 | NHCOCH₂OPh(4-OMe) | 387.38 | 99.2 | 3.81 |
| SF1771-000 | NHCOCH₂OPh(4-Me) | 371.40 | 99.2 | 4.1 |
| SF1772-000 | NHCOCH₂OPh(2-iPr) | 399.45 | 99.4 | 4.5 |
| SF1773-000 | NHSO₂Ph | 363.39 | 98.9 | 3.5 |
| SF1774-000 | NHSO₂Ph(4-NO₂) | 408.39 | 98.6 | 3.7 |
| SF1777-000 | NHCOPh | 327.33 | 99.6 | 3.7 |
| SF1779-000 | NHCOPh(4-Me) | 341.36 | 99.8 | 3.9 |
| SF1780-000 | NHSO₂Ph(4-tBu) | 419.50 | 98 | 4.3 |
| SF1784-000 | NHCOPh(2-NO₂) | 408.39 | 98.6 | 3.6 |
| SF1785-000 | NHCO(CH₂)₃Ph | 369.42 | 97.9 | 4.1 |
| SF1786-000 | NHCOCO₂Et | 323.31 | 99.8 | 3.3 |
| SF1787-000 | NHCOCH₂OPh(2-OMe) | 387.40 | 94.7 | 3.8 |
| SF1788-000 | NHCOCH₂OPh(3-OMe) | 387.40 | 96.9 | 3.9 |
| SF1789-000 | NH₂ | 223.23 | 99 | 2.97 |
| SF1790-000 | NHCOCH₂OPh(4-Cl) | 391.81 | 99.2 | 4.1 |
| SF1796-000 | NHCOCH₂OPh(2-NO₂) | 402.37 | 99 | 3.9 |
| SF1798-000 | NHSO₂-(2-thophene) | 369.42 | 93.6 | 3.5 |
| SF1799-000 | NHCO-(2-thophene) | 333.37 | 99.4 | 3.6 |
| SF1800-000 | NHCH₂Ph | 313.36 | 98 | 4.1 |
| SF1801-000 | NHSO₂Ph(3,5-diMe) | 391.45 | 95.3 | 3.9 |
| SF1803-000 | NHCOPh(4-tBu) | 383.45 | 82.99 | 4.6 |
| SF1804-000 | NHCO(CH₂)₂CO₂H | 323.31 | 98.64 | 2.7 |
| SF1806-000 | NHCOCO₂H | 295.25 | 98.08 | 4.4 |
| SF1807-000 | NHCH₂CO₂CH₂Ph | 371.4 | 93.88 | 4.1 |
| SF1808-000 | NHCO₂Et | 295.30 | 96.22 | 3.5 |
| SF1809-000 | NHCHOPh(4-Br) | 406.24 | 96.94 | 4.2 |
| SF1811-000 | NCOCH₂OPh(3-CO₂Et) | 429.42 | 98.64 | 4.07 |

+ Commercially available so HPLC data not determined

### EXAMPLE 41

### Isatin Based PTEN Inhibitors

5-nitroindoline-2,3-dione was obtained from Aldrich. Reduction of the nitro group to amino group was performed and the amino group acylated with a number of acid chlorides. The PTEN inhibition of these compounds is shown below:

TABLE 27

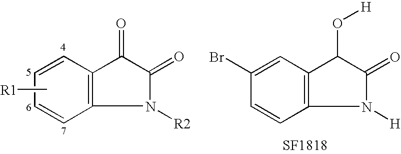

| | | | | % | |
|---|---|---|---|---|---|
| | | | Mol. | Inhibition | |
| SF Number | R1 | R2 | Wt. | @250 uM | Ret. Time |
| SF1770-000 | 5-NO₂ | H | 327.33 | 99.4 | No Data+ |
| SF1778-000 | 5-(NHCOPh(4-Me)) | H | 280.28 | 98.5 | 2.81 |
| SF1781-000 | 5-(NHCOCH₂OPh) | H | 296.3 | 97.8 | 2.76 |
| SF1783-000 | 5-NO₂ | CH₂Ph(2,4-diCl) | 351.2 | 97.8 | No Data+ |
| SF1791-000 | 5-H | CH₂Ph(4-Me) | 251 | 5.7 | No Data+ |
| SF1792-000 | 5-H | SO₂Ph(4-F) | 305 | −6.4 | No Data+ |
| SF1793-000 | 5-Me | CH₂Ph(4-Cl) | 285 | 9 | No Data+ |
| SF1794-000 | 5-iPr | H | 189 | 21.3 | No Data+ |
| SF1795-000 | 5-Br | Et | 254 | 94.3 | No Data+ |
| SF1812-000 | 5-NHCO(CH₂)₂Ph(2-NO₂) | H | 339.3 | 40.48 | 2.55 |
| SF1813-000 | 5-NHCOCH₂Cl | H | 237.63 | 1.1 | 1.70 |
| SF1814-000 | 5-Br | H | 226 | 97.6 | No Data+ |
| SF1815-000 | 5-H | H | 147.1 | 26.3 | No Data+ |
| SF1816-000 | 5-Cl | H | 181.6 | 95.01 | No Data+ |
| SF1817-000 | 5-OMe | H | 177.2 | 47.83 | No Data+ |
| SF1819-000 | 5-I | H | 273 | 96.01 | No Data+ |
| SF1820-000 | 5-F | H | 165.1 | 94.61 | No Data+ |
| SF1821-000 | 5-H | Me | 161.2 | 12 | No Data+ |
| SF1822-000 | 4,7-diCi | H | 216 | 97.31 | No Data+ |

| | | | | |
|---|---|---|---|---|
| SF1818-000 | See structure above | 242 | 97.81 | No Data+ |

+Commercially available (Bionet Research)

3. General Procedure for the Synthesis of 5-Substituted Isatins.

Hydrogenation of 5-nitroindoline-2,3-dione (Aldrich) using 10% Pd/Carbon in Methanol over hydrogen gas produced the corresponding 5-aminoindoline-2,3-dione. This product was filtered using celite, concentrated in vacuo and used without further purification. Equal stoichiometry of amine, diisopropylethyl amine, and the respective acid chloride in DMF was stirred overnight and purified by HPLC. The product purity was confirmed by electrospray LC-MS using method A. The substituted phenoxy acetyl chloride used to make SF1781 was synthesized from the corresponding substituted phenol and chloroacetic acid by methods based on those reported in the literature (Vogel, A. I.; Furniss, B. S.; Vogel, A. I., Vogel's Textbook of practical organic chemistry. 5th ed.; Longman Scientific & Technical: New York, 1989; p xxviii, 1514).

### EXAMPLE 42

### PTEN Inhibitors Based on Various Mono- and Di-Ketones

To determine the role the diketone functionality played in inhibiting PTEN activity, a variety of mono- and di-carbonyl compounds were examined and shown to have significant PTEN activity at 250 uM. These compounds all exhibited low double-digit micromolar IC50 activity in the PTEN assay as shown in the table below:

TABLE 28

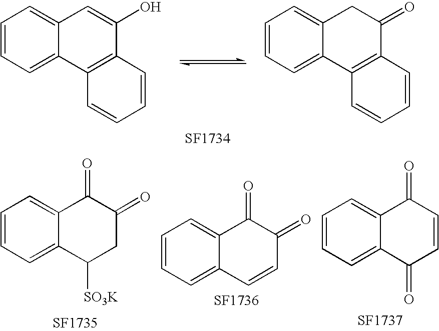

| Compound | PTEN IC50 |
|---|---|
| SF1734 | 33 uM |
| SF1735 | 20 uM |
| SF1736 | 21 uM |
| SF1737 | 43 uM |

### EXAMPLE 43

### IC50 Values for Preferred PTEN Inhibitors

Various of the disclosed PTEN inhibitors were evaluated further to determine both IC50 with PTEN and IC50 in another phosphatase, PTP1B. Values presented represent the IC50 found from one assay run and where a range of values is shown that represents the range of values from more than one experiment.

TABLE 29

| | PTEN IC50 | PTP1B IC50 |
|---|---|---|
| SF Number | (uM) | (uM) |
| SF1523-000 | 135 | 9.5 |
| SF1581-000 | 190 | N.A. |
| SF1589-000 | 60 | 8 |
| SF1609-000 | 80-113 | 5.8 |
| SF1617-000 | 66-84 | 13.6 |
| SF1668-101 | 0.276 | 0.103 |
| SF1670-000 | 2 | 17 |
| SF1672-100 | NYT | 7 |
| SF1674-101 | 0.091 | 0.045 |
| SF1675-101 | 0.111 | 0.082 |
| SF1678-101 | .357 | 0.084 |
| SF1681-000 | 50 | 50 |
| SF1704-000 | 220 | N.A. |
| SF1708-000 | 50 | 60 |
| SF1720-000 | 5 | >250 |
| SF1721-000 | 3.4 | 17 |
| SF1722-000 | 4 | 10 |
| SF1734-000 | 33 | N.A. |
| SF1735-000 | 20 | >250 |
| SF1736-000 | 21 | N.A. |
| SF1737-000 | 43 | N.A. |
| SF1740-000 | 0.3-0.4 | 1.4 |
| SF1751-000 | 0.3 | 3 |
| SF1770-000 | 2.2 | 16.3 |
| SF1771-000 | 0.395 | 0.442 |
| SF1772-000 | 0.435 | 0.681 |
| SF1773-000 | 0.221-0.297 | 0.2053 |
| SF1774-000 | 0.214 | 0.3307 |
| SF1777-000 | 0.292 | 0.558 |
| SF1778-000 | 22.05 | 11.036 |
| SF1779-000 | 0.342 | 0.233 |
| SF1780-000 | 0.269 | 0.1203 |
| SF1781-000 | 18.9 | 0.2942 |
| SF1783-000 | 4.6 | 1.1 |
| SF1784-000 | 0.599 | 1-10 |
| SF1785-000 | 5 | 1-10 |
| SF1786-000 | 0.692 | <1 |
| SF1787-000 | 0.548 | <1 |
| SF1788-000 | 0.41 | <1 |
| SF1789-000 | 0.776 | 2.97 |
| SF1790-000 | 2.6 | 1-10 |
| SF1796-000 | 0.650 | <10 |
| SF1797-000 | 4.20 | <10 |
| SF1798-000 | 0.3689 | <10 |
| SF1799-000 | 0.733 | <10 |
| SF1800-000 | 0.862 | <10 |
| SF1801-000 | 1.01 | <10 |
| SF1803-000 | 14.56 | <1 |
| SF1804-000 | 54.7 | 1-10 |
| SF1806-000 | 12.1 | 1-10 |
| SF1807-000 | NYT | <1 |
| SF1808-000 | NYT | <1 |
| SF1809-000 | NYT | 1-10 |
| SF1811-000 | NYT | 10 |
| SF1812-000 | NYT | >10 |
| SF1813-000 | NYT | >10 |
| SF1814-000 | 44.93 | 10-100 |
| SF1816-000 | 80.7 | 10-100 |
| SF1818-000 | 31.7 | <10 |
| SF1819-000 | 33.6 | <10 |
| SF1820-000 | 61.9 | 10-100 |
| SF1822-000 | 24.5 | 10 |

NYT = Not yet tested

These results indicate a number of the PTEN inhibitors disclosed herein have potent PTEN inhibition activity. These results also show significant selectivity for PTEN inhibition over PTP1B phosphatase for a number of the disclosed PTEN inhibitors.

### EXAMPLE 44

### Demonstration of RAC Activation Using PTEN Inhibitors

Mouse embryo fibroblasts (MEF) from wild type PTEN +/+ animals were pretreated with PTEN inhibitors, SF1670 (0.125 μM, 0.25 μM and 0.5 μM) and SF1740 (1 μM and 3 μM) for 30 minutes at 37° C. Following treatment, cells were stimulated in vitronectin coated (20 μg/ml) 10 cm non-tissue culture petri dish for 15 minutes at 37° C. Cell lysates were prepared in 25 mM HEPES, pH 7.5, 150 mM NaCl, 1% Igepal CA630, 10 mM MgC₁₂, 1 mM EDTA, 10% glycerol, 10 μg/ml leupeptin, 10 μg/ml aprotinin, 25 mM sodium fluoride and 1 mM sodium orthovanadate. RAC1-GTP activity reaction was measured by adding 12 μl of PAK1 agarose (GST fusion protein, corresponding to p21 binding of CRIB domain, PBD of human PAK1, expressed in E. coli and bound to glutathione agarose) to each sample and incubated at 4° C. for 45 minutes. Beads were washed three times with lysis buffer, re-suspended in 2× Laemmli sample buffer and resolved in 12% SDS-PAGE. Total RAC1 was immunoblotted to assure equal quantity of total RAC was present in the cell lysates.

RAC is instrumental to the chemotactic process and is usually activated at the leading edge of a moving cell. The biochemical results from the immunoblot described above demonstrate high level of GTPRAC in −/− MEFs compared to +/+ MEFs confirming that PTEN regulates RACGTP levels in these cells under conditions of integrin stimulation. The SF1670 at 0.125 uM and SF1740 at 1 and 3 uM concentration augmented integrin induced activation of RAC to its GTP bound state. The levels of total RAC protein in the lysates used in the binding assay were similar levels. From these data we conclude that the PTEN inhibitors SF1670 and SF1740 inhibit the capacity of PTEN phosphatase to downregulate activation state of RAC GTPase a known mediator of cell migration hence this biochemical data directly correlates with the PTEN inhibitors capacity to regulate integrin dependent cell migration on vitronectin, a known PTEN regulated cellular process.

### EXAMPLE 45

### Effect of PTEN Inhibitors on ₚAkt Levels

The ability of SF1740 to inhibit PTEN function was tested in an in vitro system using PTEN positive or negative mouse embryo fibroblasts (MEF). Cells were pre-incubated with SF1740 at different concentrations ranging from 2 to 0.125 μM for 2 hours followed by stimulation with IGF-1 for 30 minutes. Cells were then harvested and analyzed by Western Blotting for activation of Akt, which is regulated by PTEN upstream of the signaling pathway. While the phosphorylated Akt level is similarly high in PTEN knock-out MEFs, SF1740 at the 2 highest concentrations (2 and 1 μM) resulted in an increase in the expression of phospho-Akt compared with the control in PTEN positive MEFs. This demonstrates that inhibition of PTEN with a small molecule can activate Akt in a cell.

### EXAMPLE 46

### Use of PTEN Inhibitor for Sensitizing Tumor Cells

Small molecule PTEN inhibitors are administered to patients suffering from cancer via a route of administration including, but not limited to, oral, i.v., sub-cutaneous, i.v. drip, intramuscular, nasally as aerosol, dermal patch, mucous exposure, etc as compatible conventional formulations or as drug delivery modalities such as slow release formulations, depots, liposomes, microparticles, nanoparticles, and degradable and/or targeted versions thereof. The inhibitors are administered for a limited period of time sufficient to convert at least 10% of cancer cells from basal levels of phospho-Akt to at least 10% increased levels of phospho-Akt.

The patients are then withheld from further treatment with PTEN inhibitors and subsequently treated with inhibitors of the PI3 Kinase pathway including, but not limited to, singly or in combination: a) growth factor regulators and growth factor receptor inhibitors (such as antibodies and/or receptor trysine kinase inhibitors-Irressa); b) PI3 kinase inhibitors (including for examples specific isoforms, e.g. p110alpha isoform) such as but not limited to LY294002 (and prodrugs thereof as described in U.S. patent application Ser. No. 10/818,145, which is incorporated by reference), wortmanin, and other known inhibitors (such as disclosed by Piramed); c) PDK inhibitors; d) Akt inhibitors; e) mTOR inhibitors (such as but not limited to rapamycin, CCI-779, etc); f) mdm2 inhibitors; g) nfkb inhibitors; h) integrin antagonists; i) proteosome inhibitors; j) tyrosine kinase inhibitors; k) HIF inhibitors; l) and the like. As an alternative to treatment using the PI3 kinase pathway inhibitor, any single or combination of chemotherapy or radiation therapy or immunotherapy or other oncology methodology is used to affect the survival or viability or reproduction ability of the cancer cells and cancer stem cells.

In order to minimize toxicity to normal cell, the patients may be treated as described above except the administration of the PTEN inhibitor and the PI3 Kinase pathway inhibitor may overlap to a small extent.

1. A method of protecting a patient from one or more treatments that trigger apoptosis comprising administering to the patient a composition comprising a pharmaceutically acceptable amount of a PTEN inhibitor selected from the group consisting of compounds I-XIV.

2. The method of claims 1, wherein the treatment is a cancer treatment.

3. The method of claim 2, wherein the PTEN inhibitor is administered prior to, together with, or after a treatment for the cancer.

4. The method of claim 2, wherein the treatment is chemotherapy or radiation therapy.

5. A method of treating a patient suffering from damage to normal tissue attributable to stress, comprising administering to the patient a composition comprising a pharmaceutically acceptable amount of a PTEN inhibitor selected from the group consisting of compounds I-XIV.

6. The method of claim 5, wherein the PTEN inhibitor is administered prior to, together with, or after a treatment for a disease suffered by the patient.

7. A method of sensitizing cancer cells to an inhibitor of the PI3 kinase pathway comprising administering to a patient in need of such treatment a PTEN inhibitor selected from the group consisting of compounds I-XIV.

8. A method of treating apoptosis associated with a medical procedure comprising administering to a patient in need of such treatment a PTEN inhibitor selected from tie group consisting of compounds I-XIV.

9. A method of protecting a patient from radiation or chemical exposure comprising administering to the patient a composition comprising a pharmaceutically acceptable amount of a PTEN inhibitor selected from the group consisting of compounds I-XIV.

10. The method of claim 9 wherein the PTEN inhibitor is administered prior to, together with, or after the said radiation or chemical exposure.

11. A method of sensitizing cancer stem cells by exposing said cells to a PTEN inhibitor selected from the group consisting of compounds I-XIV.

12. The method of claim 9 wherein the PTEN inhibitor is administered prior to, together with, or after a treatment for a disease suffered by the patient.
